SEQUENCE LISTING (234 sequences)

SEQ ID NO: 1 - protein, 12 aa, artificial sequence
  synthetic construct
RVPNKTVTVDGA

SEQ ID NO: 2 - protein, 12 aa, artificial sequence
  Synthetic construct
DRHKSKYSSTKS

SEQ ID NO: 3 - protein, 12 aa, artificial sequence
  Synthetic construct
KNFPQQKEFPLS

SEQ ID NO: 4 - protein, 12 aa, artificial sequence
  Synthetic construct
QRNSPPAMSRRD

SEQ ID NO: 5 - protein, 12 aa, artificial sequence
  Synthetic construct
TRKPNMPHGQYL

SEQ ID NO: 6 - protein, 12 aa, artificial sequence
  Synthetic construct
KPPHLAKLPFTT

SEQ ID NO: 7 - protein, 12 aa, artificial sequence
  Synthetic construct
NKRPPTSHRIHA

SEQ ID NO: 8 - protein, 12 aa, artificial sequence
  Synthetic construct
NLPRYQPPCKPL

SEQ ID NO: 9 - protein, 12 aa, artificial sequence
  Synthetic construct
RPPWKKPIPPSE

SEQ ID NO: 10 - protein, 12 aa, artificial sequence
  Synthetic construct
RQRPKDHFFSRP

SEQ ID NO: 11 - protein, 12 aa, artificial sequence
  Synthetic construct
SVPNKXVTVDGX

SEQ ID NO: 12 - protein, 12 aa, artificial sequence
  Synthetic construct
TTKWRHRAPVSP

SEQ ID NO: 13 - protein, 12 aa, artificial sequence
  Synthetic construct
WLGKNRIKPRAS

SEQ ID NO: 14 - protein, 12 aa, artificial sequence
  Synthetic construct
SNFKTPLPLTQS

SEQ ID NO: 15 - protein, 12 aa, artificial sequence
  Synthetic construct
SVSVGMKPSPRP

SEQ ID NO: 16 - protein, 7 aa, artificial sequence
  Synthetic construct
DLHTVYH

SEQ ID NO: 17 - protein, 7 aa, artificial sequence
  Synthetic construct
HIKPPTR

SEQ ID NO: 18 - protein, 7 aa, artificial sequence
  Synthetic construct
HPVWPAI

SEQ ID NO: 19 - protein, 7 aa, artificial sequence
  Synthetic construct
MPLYYLQ

SEQ ID NO: 20 - protein, 26 aa, artificial sequence
  Synthetic construct
HLTVPWRGGGSAVPFYSHSQITLPNH

SEQ ID NO: 21 - protein, 41 aa, artificial sequence
  Synthetic construct
GPHDTSSGGVRPNLHHTSKKEKRENRKVPFYSHSVTSRGNV

SEQ ID NO: 22 - protein, 7 aa, artificial sequence
  Synthetic construct
KHPTYRQ

SEQ ID NO: 23 - protein, 7 aa, artificial sequence
  Synthetic construct
HPMSAPR

SEQ ID NO: 24 - protein, 7 aa, artificial sequence
  Synthetic construct
MPKYYLQ

SEQ ID NO: 25 - protein, 7 aa, artificial sequence
  Synthetic construct
MHAHSIA

SEQ ID NO: 26 - protein, 12 aa, artificial sequence
  Synthetic construct
AKPISQHLQRGS

SEQ ID NO: 27 - protein, 12 aa, artificial sequence
  Synthetic construct
APPTPAAASATT

SEQ ID NO: 28 - protein, 12 aa, artificial sequence
  Synthetic construct
DPTEGARRTIMT

SEQ ID NO: 29 - protein, 12 aa, artificial sequence
  Synthetic construct
LDTSFPPVPFHA

SEQ ID NO: 30 - protein, 12 aa, artificial sequence
  Synthetic construct
LDTSFHQVPFHQ

SEQ ID NO: 31 - protein, 11 aa, artificial sequence
  Synthetic construct
LPRIANTWSPS

SEQ ID NO: 32 - protein, 12 aa, artificial sequence
  Synthetic construct
RTNAADHPAAVT

SEQ ID NO: 33 - protein, 12 aa, artificial sequence
  Synthetic construct
SLNWVTIPGPKI

SEQ ID NO: 34 - protein, 12 aa, artificial sequence
  Synthetic construct
TDMQAPTKSYSN

SEQ ID NO: 35 - protein, 12 aa, artificial sequence
  Synthetic construct
TIMTKSPSLSCG

SEQ ID NO: 36 - protein, 12 aa, artificial sequence
  Synthetic construct
TPALDGLRQPLR

SEQ ID NO: 37 - protein, 12 aa, artificial sequence
  Synthetic construct
TYPASRLPLLAP

SEQ ID NO: 38 - protein, 12 aa, artificial sequence
  Synthetic construct
AKTHKHPAPSYS

SEQ ID NO: 39 - protein, 12 aa, artificial sequence
  Synthetic construct
TDPTPFSISPER

SEQ ID NO: 40 - protein, 12 aa, artificial sequence
  Synthetic construct
SQNWQDSTSYSN

SEQ ID NO: 41 - protein, 12 aa, artificial sequence
  Synthetic construct
WHDKPQNSSKST

SEQ ID NO: 42 - protein, 12 aa, artificial sequence
  Synthetic construct
LDVESYKGTSMP

SEQ ID NO: 43 - protein, 7 aa, artificial sequence
  Synthetic construct
NTPKENW

SEQ ID NO: 44 - protein, 7 aa, artificial sequence
  Synthetic construct
NTPASNR

SEQ ID NO: 45 - protein, 7 aa, artificial sequence
  Synthetic construct
PRGMLST

SEQ ID NO: 46 - protein, 7 aa, artificial sequence
  Synthetic construct
PPTYLST

SEQ ID NO: 47 - protein, 12 aa, artificial sequence
  Synthetic construct
TIPTHRQHDYRS

SEQ ID NO: 48 - protein, 7 aa, artificial sequence
  Synthetic construct
TPPTHRL

SEQ ID NO: 49 - protein, 7 aa, artificial sequence
  Synthetic construct
LPTMSTP

SEQ ID NO: 50 - protein, 7 aa, artificial sequence
  Synthetic construct
LGTNSTP

SEQ ID NO: 51 - protein, 12 aa, artificial sequence
  Synthetic construct
TPLTGSTNLLSS

SEQ ID NO: 52 - protein, 7 aa, artificial sequence
  Synthetic construct
TPLTKET

SEQ ID NO: 53 - protein, 7 aa, artificial sequence
  Synthetic construct
KQSHNPP

SEQ ID NO: 54 - protein, 7 aa, artificial sequence
  Synthetic construct
QQSHNPP

SEQ ID NO: 55 - protein, 7 aa, artificial sequence
  Synthetic construct
TQPHNPP

SEQ ID NO: 56 - protein, 12 aa, artificial sequence
  Synthetic construct
STNLLRTSTVHP

SEQ ID NO: 57 - protein, 12 aa, artificial sequence
  Synthetic construct
HTQPSYSSTNLF

SEQ ID NO: 58 - protein, 7 aa, artificial sequence
  Synthetic construct
SLLSSHA

SEQ ID NO: 59 - protein, 12 aa, artificial sequence
  Synthetic construct
QQSSISLSSHAV

SEQ ID NO: 60 - protein, 7 aa, artificial sequence
  Synthetic construct
NASPSSL

SEQ ID NO: 61 - protein, 7 aa, artificial sequence
  Synthetic construct
HSPSSLR

SEQ ID NO: 62 - protein, 7 aa, artificial sequence
  Synthetic construct
KXSHHTH

SEQ ID NO: 63 - protein, 7 aa, artificial sequence
  Synthetic construct
EXSHHTH

SEQ ID NO: 64 - protein, 12 aa, artificial sequence
  Synthetic construct
SHHTHYGQPGPV

SEQ ID NO: 65 - protein, 7 aa, artificial sequence
  Synthetic construct
LESTSLL

SEQ ID NO: 66 - protein, 7 aa, artificial sequence
  Synthetic construct
DLTLPFH

SEQ ID NO: 67 - protein, 8 aa, artificial sequence
  Synthetic construct
RTNAADHP

SEQ ID NO: 68 - protein, 12 aa, artificial sequence
  Synthetic construct
IPWWNIRAPLNA

SEQ ID NO: 69 - protein, 18 aa, artificial sequence
  Synthetic construct
EQISGSLVAAPWEGEGER

SEQ ID NO: 70 - protein, 12 aa, artificial sequence
  synthetic construct
TPPELLHGAPRS

SEQ ID NO: 71 - protein, 18 aa, artificial sequence
  Synthetic construct
LDTSFHQVPFHQKRKRKD

SEQ ID NO: 72 - protein, 18 aa, artificial sequence
  Synthetic construct
EQISGSLVAAPWKRKRKD

SEQ ID NO: 73 - protein, 18 aa, artificial sequence
  Synthetic construct
TPPELLHGDPRSKRKRKD

SEQ ID NO: 74 - protein, 13 aa, artificial sequence
  Synthetic construct
NTSQLSTEGEGED

SEQ ID NO: 75 - protein, 13 aa, artificial sequence
  Synthetic construct
TPPELLHGDPRSC

SEQ ID NO: 76 - protein, 20 aa, artificial sequence
  synthetic construct
HINKTNPHQGNHHSEKTQRQ

SEQ ID NO: 77 - protein, 15 aa, artificial sequence
  Synthetic construct
HAHKNQKETHQRHAA

SEQ ID NO: 78 - protein, 15 aa, artificial sequence
  Synthetic construct
HEHKNQKETHQRHAA

SEQ ID NO: 79 - protein, 20 aa, artificial sequence
  Synthetic construct
HNHMQERYTEPQHSPSVNGL

SEQ ID NO: 80 - protein, 17 aa, artificial sequence
  Synthetic construct
THSTHNHGSPRHTNADA

SEQ ID NO: 81 - protein, 20 aa, artificial sequence
  synthetic construct
GSCVDTHKADSCVANNGPAT

SEQ ID NO: 82 - protein, 20 aa, artificial sequence
  synthetic construct
AQSQLPDKHSGLHERAPQRY

SEQ ID NO: 83 - protein, 20 aa, artificial sequence
  Synthetic construct
AQSQLPAKHSGLHERAPQRY

SEQ ID NO: 84 - protein, 20 aa, artificial sequence
  Synthetic construct
AQSQLPEKHSGLHERAPQRY

SEQ ID NO: 85 - protein, 20 aa, artificial sequence
  synthetic construct
TDMMHNHSDNSPPHRRSPRN

SEQ ID NO: 86 - protein, 20 aa, artificial sequence
  synthetic construct
TPPELAHTPHHLAQTRLTDR

SEQ ID NO: 87 - protein, 12 aa, artificial sequence
  Synthetic construct
RLLRLLRLLRLL

SEQ ID NO: 88 - protein, 12 aa, artificial sequence
  Synthetic construct
TPPELLHGEPRS

SEQ ID NO: 89 - protein, 12 aa, artificial sequence
  Synthetic construct
TPPELLHGAPRS

SEQ ID NO: 90 - protein, 12 aa, artificial sequence
  Synthetic construct
EQISGSLVAAPW

SEQ ID NO: 91 - protein, 12 aa, artificial sequence
  Synthetic construct
NEVPARNAPWLV

SEQ ID NO: 92 - protein, 13 aa, artificial sequence
  Synthetic construct
NSPGYQADSVAIG

SEQ ID NO: 93 - protein, 12 aa, artificial sequence
  Synthetic construct
AKPISQHLQRGS

SEQ ID NO: 94 - protein, 12 aa, artificial sequence
  Synthetic construct
LDTSFPPVPFHA

SEQ ID NO: 95 - protein, 12 aa, artificial sequence
  Synthetic construct
SLNWVTIPGPKI

SEQ ID NO: 96 - protein, 12 aa, artificial sequence
  Synthetic construct
TQDSAQKSPSPL

SEQ ID NO: 97 - protein, 12 aa, artificial sequence
  Synthetic construct
KELQTRNVVQRE

SEQ ID NO: 98 - protein, 12 aa, artificial sequence
  Synthetic construct
QRNSPPAMSRRD

SEQ ID NO: 99 - protein, 12 aa, artificial sequence
  Synthetic construct
TPTANQFTQSVP

SEQ ID NO: 100 - protein, 12 aa, artificial sequence
  Synthetic construct
AAGLSQKHERNR

SEQ ID NO: 101 - protein, 12 aa, artificial sequence
  Synthetic construct
ETVHQTPLSDRP

SEQ ID NO: 102 - protein, 12 aa, artificial sequence
  Synthetic construct
KNFPQQKEFPLS

SEQ ID NO: 103 - protein, 12 aa, artificial sequence
  Synthetic construct
LPALHIQRHPRM

SEQ ID NO: 104 - protein, 12 aa, artificial sequence
  Synthetic construct
QPSHSQSHNLRS

SEQ ID NO: 105 - protein, 12 aa, artificial sequence
  Synthetic construct
RGSQKSKPPRPP

SEQ ID NO: 106 - protein, 12 aa, artificial sequence
  Synthetic construct
THTQKTPLLYYH

SEQ ID NO: 107 - protein, 12 aa, artificial sequence
  Synthetic construct
TKGSSQAILKST

SEQ ID NO: 108 - protein, 7 aa, artificial sequence
  Synthetic construct
TAATTSP

SEQ ID NO: 109 - protein, 7 aa, artificial sequence
  Synthetic construct
LGIPQNL

SEQ ID NO: 110 - protein, 20 aa, artificial sequence
  Synthetic construct
THSTHNHGSPRHTNADAGNP

SEQ ID NO: 111 - protein, 20 aa, artificial sequence
  Synthetic construct
QQHKVHHQNPDRSTQDAHHS

SEQ ID NO: 112 - protein, 15 aa, artificial sequence
  Synthetic construct
HHGTHHNATKQKNHV

SEQ ID NO: 113 - protein, 15 aa, artificial sequence
  Synthetic construct
STLHKYKSQDPTPHH

SEQ ID NO: 114 - protein, 12 aa, artificial sequence
  Synthetic construct
SVSVGMKPSPRP

SEQ ID NO: 115 - protein, 12 aa, artificial sequence
  synthetic construct
TPPTNVLMLATK

SEQ ID NO: 116 - protein, 12 aa, artificial sequence
  Synthetic construct
TPPELLHGDPRS

SEQ ID NO: 117 - protein, 7 aa, artificial sequence
  synthetic construct
NTSQLST

SEQ ID NO: 118 - protein, 15 aa, artificial sequence
  Synthetic construct
STLHKYKSQDPTPHH

SEQ ID NO: 119 - protein, 12 aa, artificial sequence
  synthetic construct
GMPAMHWIHPFA

SEQ ID NO: 120 - protein, 15 aa, artificial sequence
  synthetic construct
HDHKNQKETHQRHAA

SEQ ID NO: 121 - protein, 20 aa, artificial sequence
  Synthetic construct
HNHMQERYTDPQHSPSVNGL

SEQ ID NO: 122 - protein, 20 aa, artificial sequence
  synthetic construct
TAEIQSSKNPNPHPQRSWTN

SEQ ID NO: 123 - protein, 12 aa, artificial sequence
  Synthetic construct
KRGRHKRPKRHK

SEQ ID NO: 124 - protein, 7 aa, artificial sequence
  Synthetic construct
RLLRLLR

SEQ ID NO: 125 - protein, 12 aa, artificial sequence
  Synthetic construct
HKPRGGRKKALH

SEQ ID NO: 126 - protein, 18 aa, artificial sequence
  Synthetic construct
KPRPPHGKKHRPKHRPKK

SEQ ID NO: 127 - protein, 18 aa, artificial sequence
  Synthetic construct
RGRPKKGHGKRPGHRARK

SEQ ID NO: 128 - protein, 12 aa, artificial sequence
  Synthetic construct
TPFHSPENAPGS

SEQ ID NO: 129 - protein, 13 aa, artificial sequence
  Synthetic construct
TPFHSPENAPGSK

SEQ ID NO: 130 - protein, 16 aa, artificial sequence
  Synthetic construct
TPFHSPENAPGSGGGS

SEQ ID NO: 131 - protein, 17 aa, artificial sequence
  Synthetic construct
TPFHSPENAPGSGGGSS

SEQ ID NO: 132 - protein, 15 aa, artificial sequence
  Synthetic construct
TPFHSPENAPGSGGG

SEQ ID NO: 133 - protein, 7 aa, artificial sequence
  Synthetic construct
FTQSLPR

SEQ ID NO: 134 - protein, 12 aa, artificial sequence
  Synthetic construct
KQATFPPNPTAY

SEQ ID NO: 135 - protein, 12 aa, artificial sequence
  Synthetic construct
HGHMVSTSQLSI

SEQ ID NO: 136 - protein, 7 aa, artificial sequence
  Synthetic construct
LSPSRMK

SEQ ID NO: 137 - protein, 7 aa, artificial sequence
  Synthetic construct
LPIPRMK

SEQ ID NO: 138 - protein, 7 aa, artificial sequence
  Synthetic construct
HQRPYLT

SEQ ID NO: 139 - protein, 7 aa, artificial sequence
  Synthetic construct
FPPLLRL

SEQ ID NO: 140 - protein, 7 aa, artificial sequence
  Synthetic construct
QATFMYN

SEQ ID NO: 141 - protein, 11 aa, artificial sequence
  Synthetic construct
VLTSQLPNHSM

SEQ ID NO: 142 - protein, 7 aa, artificial sequence
  Synthetic construct
HSTAYLT

SEQ ID NO: 143 - protein, 12 aa, artificial sequence
  Synthetic construct
APQQRPMKTFNT

SEQ ID NO: 144 - protein, 12 aa, artificial sequence
  Synthetic construct
APQQRPMKTVQY

SEQ ID NO: 145 - protein, 7 aa, artificial sequence
  Synthetic construct
PPWLDLL

SEQ ID NO: 146 - protein, 7 aa, artificial sequence
  Synthetic construct
PPWTFPL

SEQ ID NO: 147 - protein, 7 aa, artificial sequence
  Synthetic construct
SVTHLTS

SEQ ID NO: 148 - protein, 7 aa, artificial sequence
  Synthetic construct
VITRLTS

SEQ ID NO: 149 - protein, 12 aa, artificial sequence
  Synthetic construct
DLKPPLLALSKV

SEQ ID NO: 150 - protein, 12 aa, artificial sequence
  Synthetic construct
SHPSGALQEGTF

SEQ ID NO: 151 - protein, 12 aa, artificial sequence
  Synthetic construct
FPLTSKPSGACT

SEQ ID NO: 152 - protein, 12 aa, artificial sequence
  Synthetic construct
DLKPPLLALSKV

SEQ ID NO: 153 - protein, 7 aa, artificial sequence
  Synthetic construct
PLLALHS

SEQ ID NO: 154 - protein, 7 aa, artificial sequence
  Synthetic construct
VPISTQI

SEQ ID NO: 155 - protein, 12 aa, artificial sequence
  Synthetic construct
YAKQHYPISTFK

SEQ ID NO: 156 - protein, 7 aa, artificial sequence
  Synthetic construct
HSTAYLT

SEQ ID NO: 157 - protein, 12 aa, artificial sequence
  Synthetic construct
STAYLVAMSAAP

SEQ ID NO: 158 - protein, 12 aa, artificial sequence
  Synthetic construct
SVSVGMKPSPRP

SEQ ID NO: 159 - protein, 12 aa, artificial sequence
  Synthetic construct
TMGFTAPRFPHY

SEQ ID NO: 160 - protein, 12 aa, artificial sequence
  Synthetic construct
NLQHSVGTSPVW

SEQ ID NO: 161 - protein, 15 aa, artificial sequence
  Synthetic construct
QLSYHAYPQANHHAP

SEQ ID NO: 162 - protein, 12 aa, artificial sequence
  Synthetic construct
NQAASITKRVPY

SEQ ID NO: 163 - protein, 14 aa, artificial sequence
  Synthetic construct
SGCHLVYDNGFCDH

SEQ ID NO: 164 - protein, 14 aa, artificial sequence
  Synthetic construct
ASCPSASHADPCAH

SEQ ID NO: 165 - protein, 14 aa, artificial sequence
  Synthetic construct
NLCDSARDSPRCKV

SEQ ID NO: 166 - protein, 12 aa, artificial sequence
  Synthetic construct
NHSNWKTAADFL

SEQ ID NO: 167 - protein, 12 aa, artificial sequence
  Synthetic construct
GSSTVGRPLSYE

SEQ ID NO: 168 - protein, 12 aa, artificial sequence
  Synthetic construct
SDTISRLHVSMT

SEQ ID NO: 169 - protein, 12 aa, artificial sequence
  Synthetic construct
SPLTVPYERKLL

SEQ ID NO: 170 - protein, 12 aa, artificial sequence
  Synthetic construct
SPYPSWSTPAGR

SEQ ID NO: 171 - protein, 12 aa, artificial sequence
  Synthetic construct
VQPITNTRYEGG

SEQ ID NO: 172 - protein, 12 aa, artificial sequence
  Synthetic construct
WPMHPEKGSRWS

SEQ ID NO: 173 - protein, 14 aa, artificial sequence
  Synthetic construct
DACSGNGHPNNCDR

SEQ ID NO: 174 - protein, 14 aa, artificial sequence
  Synthetic construct
DHCLGRQLQPVCYP

SEQ ID NO: 175 - protein, 14 aa, artificial sequence
  Synthetic construct
DWCDTIIPGRTCHG

SEQ ID NO: 176 - protein, 12 aa, artificial sequence
  Synthetic construct
ALPRIANTWSPS

SEQ ID NO: 177 - protein, 12 aa, artificial sequence
  Synthetic construct
YPSFSPTYRPAF

SEQ ID NO: 178 - protein, 20 aa, artificial sequence
  Synthetic construct
AHPESLGIKYALDGNSDPHA

SEQ ID NO: 179 - protein, 20 aa, artificial sequence
  Synthetic construct
ASVSNYPPIHHLATSNTTVN

SEQ ID NO: 180 - protein, 14 aa, artificial sequence
  Synthetic construct
DECMEPLNAAHCWR

SEQ ID NO: 181 - protein, 14 aa, artificial sequence
  Synthetic construct
DECMHGSDVEFCTS

SEQ ID NO: 182 - protein, 14 aa, artificial sequence
  Synthetic construct
DLCSMQMMNTGCHY

SEQ ID NO: 183 - protein, 14 aa, artificial sequence
  Synthetic construct
DLCSSPSTWGSCIR

SEQ ID NO: 184 - protein, 20 aa, artificial sequence
  Synthetic construct
DPNESNYENATTVSQPTRHL

SEQ ID NO: 185 - protein, 20 aa, artificial sequence
  Synthetic construct
EPTHPTMRAQMHQSLRSSSP

SEQ ID NO: 186 - protein, 20 aa, artificial sequence
  Synthetic construct
GNTDTTPPNAVMEPTVQHKW

SEQ ID NO: 187 - protein, 15 aa, artificial sequence
  Synthetic construct
NGPDMVQSVGKHKNS

SEQ ID NO: 188 - protein, 15 aa, artificial sequence
  Synthetic construct
NGPEVRQIPANFEKL

SEQ ID NO: 189 - protein, 20 aa, artificial sequence
  Synthetic construct
NNTSADNPPETDSKHHLSMS

SEQ ID NO: 190 - protein, 20 aa, artificial sequence
  Synthetic construct
NNTWPEGAGHTMPSTNIRQA

SEQ ID NO: 191 - protein, 20 aa, artificial sequence
  Synthetic construct
NPTATPHMKDPMHSNAHSSA

SEQ ID NO: 192 - protein, 20 aa, artificial sequence
  Synthetic construct
NPTDHIPANSTNSRVSKGNT

SEQ ID NO: 193 - protein, 15 aa, artificial sequence
  Synthetic construct
NPTDSTHMMHARNHE

SEQ ID NO: 194 - protein, 14 aa, artificial sequence
  Synthetic construct
QHCITERLHPPCTK

SEQ ID NO: 195 - protein, 14 aa, artificial sequence
  Synthetic construct
TPCAPASFNPHCSR

SEQ ID NO: 196 - protein, 14 aa, artificial sequence
  Synthetic construct
TPCATYPHFSGCRA

SEQ ID NO: 197 - protein, 20 aa, artificial sequence
  Synthetic construct
WCTDFCTRSTPTSTSRSTTS

SEQ ID NO: 198 - protein, 20 aa, artificial sequence
  Synthetic construct
APPLKTYMQERELTMSQNKD

SEQ ID NO: 199 - protein, 20 aa, artificial sequence
  Synthetic construct
EPPTRTRVNNHTVTVQAQQH

SEQ ID NO: 200 - protein, 14 aa, artificial sequence
  Synthetic construct
GYCLRGDEPAVCSG

SEQ ID NO: 201 - protein, 20 aa, artificial sequence
  Synthetic construct
LSSKDFGVTNTDQRTYDYTT

SEQ ID NO: 202 - protein, 14 aa, artificial sequence
  Synthetic construct
NFCETQLDLSVCTV

SEQ ID NO: 203 - protein, 14 aa, artificial sequence
  Synthetic construct
NTCQPTKNATPCSA

SEQ ID NO: 204 - protein, 20 aa, artificial sequence
  Synthetic construct
PSEPERRDRNIAANAGRFNT

SEQ ID NO: 205 - protein, 18 aa, artificial sequence
  Synthetic construct
THNMSHFPPSGHPKRTAT

SEQ ID NO: 206 - protein, 14 aa, artificial sequence
  Synthetic construct
TTCPTMGTYHVCWL

SEQ ID NO: 207 - protein, 20 aa, artificial sequence
  Synthetic construct
YCADHTPDPANPNKICGYSH

SEQ ID NO: 208 - protein, 20 aa, artificial sequence
  Synthetic construct
AANPHTEWDRDAFQLAMPPK

SEQ ID NO: 209 - protein, 20 aa, artificial sequence
  Synthetic construct
DLHPMDPSNKRPDNPSDLHT

SEQ ID NO: 210 - protein, 14 aa, artificial sequence
  Synthetic construct
ESCVSNALMNQCIY

SEQ ID NO: 211 - protein, 20 aa, artificial sequence
  Synthetic construct
HNKADSWDPDLPPHAGMSLG

SEQ ID NO: 212 - protein, 20 aa, artificial sequence
  Synthetic construct
LNDQRKPGPPTMPTHSPAVG

SEQ ID NO: 213 - protein, 14 aa, artificial sequence
  Synthetic construct
NTCATSPNSYTCSN

SEQ ID NO: 214 - protein, 14 aa, artificial sequence
  Synthetic construct
SDCTAGLVPPLCAT

SEQ ID NO: 215 - protein, 20 aa, artificial sequence
  Synthetic construct
TIESSQHSRTHQQNYGSTKT

SEQ ID NO: 216 - protein, 20 aa, artificial sequence
  Synthetic construct
VGTMKQHPTTTQPPRVSATN

SEQ ID NO: 217 - protein, 20 aa, artificial sequence
  Synthetic construct
YSETPNDQKPNPHYKVSGTK

SEQ ID NO: 218 - protein, 8 aa, Artificial Sequence
  Caspase 3 cleavage site
LESGDEVD

SEQ ID NO: 219 - protein, 7 aa, artificial sequence
  synthetic construct
GGKGGAG

SEQ ID NO: 220 - protein, 7 aa, artificial sequence
  synthetic construct
GGAGGAG

SEQ ID NO: 221 - protein, 33 aa, artificial sequence
  synthetic construct
EPEPEPEPIPEPPKEAPVVIEKPKPKPKPKPKP

SEQ ID NO: 222 - protein, 21 aa, artificial sequence
  synthetic construct
GKGKGKGKGKGKGKGKGKGKG

SEQ ID NO: 223 - protein, 20 aa, artificial sequence
  synthetic construct
KGGGSKGGGSKGGGSKGGGS

SEQ ID NO: 224 - protein, 67 aa, artificial sequence
  synthetic construct
PNTSQLSTGGGRTNAADHPKCGGGNTSQLSTGGGRTNAADHPKCGGGNTSQLSTGGGRTNAADHPKC

SEQ ID NO: 225 - protein, 56 aa, artificial sequence
  synthetic construct
DPRTNAADHPAAVTGGGCGGGRTNAADHPAAVTGGGCGGGRTNAADHPAAVTGGGC

SEQ ID NO: 226 - protein, 51 aa, artificial sequence
  synthetic construct
DPRTNAADHPAAVTGGGCGGGIPWWNIRAPLNAGGGCGGGDLTLPFHGGGC

SEQ ID NO: 227 - protein, 58 aa, artificial sequence
  synthetic construct
RTNAADHPAAVTGGGCDPGGGRTNAADHPAAVTGGGCDPGGGRTNAADHPAAVTGGGC

SEQ ID NO: 228 - protein, 83 aa, artificial sequence
  synthetic construct
DPTPPTNVLMLATKGGGRTNAADHPKCGGGTPPTNVLMLATKGGGRTNAADHPKCGGGTPPTNVLMLATKGGGRTNAADHPKC

SEQ ID NO: 229 - protein, 83 aa, artificial sequence
  synthetic construct
DPRTNAADHPGGGTPPTNVLMLATKKCGGGRTNAADHPGGGTPPTNVLMLATKKCGGGRTNAADHPGGGTPPTNVLMLATKKC

SEQ ID NO: 230 - protein, 20 aa, artificial sequence
  synthetic construct
HDHKNQKETHQRHAAKKKKK

SEQ ID NO: 231 - protein, 25 aa, artificial sequence
  synthetic construct
AQSQLPDKHSGLHERAPQRYKKKKK

SEQ ID NO: 232 - protein, 21 aa, artificial sequence
  synthetic construct
AQSQLPDKHSGLHERAPQRYC

SEQ ID NO: 233 - protein, 21 aa, artificial sequence
  synthetic construct
TAEIQSSKNPNPHPQRSWTNC

SEQ ID NO: 234 - protein, 27 aa, artificial sequence
  synthetic construct
LRFLARRFLKRRARKWWNAWKVWVTRC

(19) United States
(12) Patent Application Publication
(10) Pub. No.: US 2011/0274639 A1
(43) Pub. Date: Nov. 10, 2011

(54) PEPTIDE-BASED COLORING REAGENTS FOR PERSONAL CARE

(75) Inventor: JOHN P. O'BRIEN, Oxford, PA (US)

(73) Assignee: E. I. DU PONT DE NEMOURS AND COMPANY, Wilmington, DE (US)

(21) Appl. No.: 12/945,969

(22) Filed: Nov. 15, 2010

Related U.S. Application Data
(60) Provisional application No. 61/261,395, filed on Nov. 16, 2009.

(51) Int. Cl. A61K 8/64; A61Q 5/00; C07K 2/00

(52) U.S. Cl. 424/70.6; 530/300

(57) ABSTRACT

Peptide-based coloring reagents are described in which a body surface-binding peptide is covalently attached to a pigment, the surface of which is coated with a layer containing at least 3 atom percent of silicon. The peptide-based coloring reagents are useful for coloring body surfaces such as hair, skin, nails, and teeth.

### FIELD OF THE INVENTION

The invention relates to the field of personal care products. More specifically, the invention relates to peptide-based coloring reagents comprising a body surface-binding peptide covalently attached to a pigment having a coating containing silicon.

### BACKGROUND OF THE INVENTION

Peptide-based coloring reagents in which specific body surface-binding peptides are coupled to a coloring reagent, such as a pigment have been described (Huang et al., U.S. Pat. No. 7,220,405). The body surface-binding peptides may be coupled to the pigment through covalent bonds (Huang et al. supra; and Rothe et al., WO 2004/000257) or non-covalent interaction (Huang et al. U.S. Patent Application Publication No. 2005/0226839). These peptide-based colorants provide an alternative to oxidative hair dyes, which may cause hair damage, and temporary hair dyes, which are removed from the hair after one shampoo, and may also be used to color other body surfaces, such as skin, nails, and teeth.

There are currently two limitations to the use of these peptide-based coloring reagents. Different chemistries may be needed to covalently attach the body surface-binding peptide to the pigment, depending on the pigment used. Additionally, it may be difficult to obtain and to retain a good dispersion of the pigment particles.

Therefore, the need exists for peptide-based coloring reagents that can be prepared using uniform chemistry and which disperse readily in aqueous solutions.

### SUMMARY OF THE INVENTION

The stated need is addressed herein by providing peptide-based coloring reagents in which a body surface-binding peptide is covalently attached to a pigment, the surface of which is coated with a layer containing at least 3 atom percent of silicon.

Accordingly, in one embodiment the invention provides a peptide-based coloring reagent selected from the group consisting of:

a) (BSBP)ₙ—CP; and

b) [(BSBP)ₘ—S]ₙ—CP;

wherein:
(i) BSBP is a body surface-binding peptide;
(ii) CP is a coated pigment containing at least 3 atom percent of silicon on its surface, as determined by electron spectroscopy for chemical analysis (ESCA);
(iii) S is a molecular spacer;
(iv) BSBP is covalently bound to the surface of CP in (a) and S is covalently bound to the surface of CP in (b);
(v) m ranges from 1 to about 50; and
(vi) n ranges from 1 to about 100,000.

In another embodiment, the invention provides a personal care composition comprising at least one peptide-based coloring reagent selected from the group consisting of:

a) (BSBP)ₙ—CP; and

b) [(BSBP)ₘ—S]ₙ—CP;

wherein:
(i) BSBP is a body surface-binding peptide;
(ii) CP is a coated pigment containing at least 3 atom percent of silicon on its surface, as determined by electron spectroscopy for chemical analysis (ESCA);
(iii) S is a molecular spacer;
(iv) BSBP is covalently bound to the surface of CP in (a) and S is covalently bound to the surface of CP in (b);
(v) m ranges from 1 to about 50; and
(vi) n ranges from 1 to about 100,000.

In another embodiment, the invention provides a method for coloring a body surface comprising: applying a personal care composition comprising at least one peptide-based coloring reagent selected from the group consisting of:

a) (BSBP)ₙ—CP; and

b) [(BSBP)ₘ—S]ₙ—CP;

wherein:
(i) BSBP is a body surface-binding peptide;
(ii) CP is a coated pigment containing at least 3 atom percent of silicon on its surface, as determined by electron spectroscopy for chemical analysis (ESCA);
(iii) S is a molecular spacer;
(iv) BSBP is covalently bound to the surface of CP in (a) and S is covalently bound to the surface of CP in (b);
(v) m ranges from 1 to about 50; and
(vi) n ranges from 1 to about 100,000;
   to the body surface for a time sufficient for the peptide-based coloring reagent to bind to the body surface.

### BRIEF DESCRIPTION OF THE BIOLOGICAL SEQUENCES

The following sequences conform with 37 C.F.R. 1.821-1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs:1-127, 230-234 are amino acid sequences of hair-binding peptides.

SEQ ID NOs:128-175 are amino acid sequences of skin-binding peptides.

SEQ ID NOs:176-177 are amino acid sequences of nail-binding peptides.

SEQ ID NOs:178-217 are amino acid sequences of tooth-binding peptides.

SEQ ID NO:218 is the amino acid sequence of the Caspase 3 cleavage site.

SEQ ID NOs:219-223 are the amino acid sequences of exemplary peptide spacers.

SEQ ID NOs:224-229 are the amino acid sequences of exemplary hair-binding domains.

### DETAILED DESCRIPTION OF THE INVENTION

Disclosed herein are peptide-based coloring reagents in which body surface-binding peptides are covalently attached to a coated pigment. The pigment is coated with silica and/or a silane reagent. The silane coupling chemistry provides a universal coupling chemistry for covalently attaching body surface-binding peptides to the surface of pigment particles. Additionally, the coated pigments are readily dispersed in aqueous solutions.

The peptide-based coloring reagents disclosed herein are useful for personal care compositions for coloring body surfaces such as hair, skin, nails, and teeth.

### Definitions

The following definitions are used herein and should be referred to for interpretation of the claims and the specification.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

The term “invention” or “present invention” as used herein is a non-limiting term and is not intended to refer to any single embodiment of the particular invention but encompasses all possible embodiments as described in the specification and the claims.

As used herein, the terms “polypeptide” and “peptide” are used interchangeably to refer to a polymer of two or more amino acids joined together by a peptide bond. In one aspect, this term also includes post expression modifications of the polypeptide, for example, glycosylations, acetylations, phosphorylations and the like. Included within the definition are, for example, peptides containing one or more analogues of an amino acid or labeled amino acids and peptidomimetics. In one embodiment, the peptides are comprised of L-amino acids.

The term “body surface-binding peptide”, also referred to herein as “BSBP”, refers to a peptide that binds with high affinity to at least one body surface. The term body surface-binding peptide may include single “fingers” of about 7-60 amino acids that define a single domain having binding affinity for a body surface. Examples of these fingers are provided in Table A. Alternatively the body surface-binding peptide may encompass body surface-binding fingers which are linked together to form body surface-binding domains (referred to herein as “hands”). In one embodiment, the body surface-binding peptide is selected from the group consisting of hair-binding peptides, skin-binding peptides, nail-binding peptides, and oral cavity surface-binding peptides, such as a tooth-binding peptide. In a preferred embodiment, the body surface-binding peptide is selected from the group consisting of hair-binding peptides, skin-binding peptides, nail-binding peptides, and tooth-binding peptides.

As used herein, the terms “body surface hand” and “body surface-binding domain” refer to a single chain peptide comprising at least two body surface-binding peptides linked together by an optional molecular spacer, wherein the inclusion of a molecular spacer is preferred. In one embodiment, the molecular spacer is a peptide linker.

The term “body surface” refers to any surface of the human body that may serve as a substrate for the binding of a peptide-based coloring reagent. Typical body surfaces include but are not limited to hair, skin, nails, teeth, and tissues of the oral cavity, such as gums.

The term “peptide-based coloring reagent” refers to a coloring reagent comprising a body surface-binding peptide attached by a covalent bond to a pigment, the surface of which is coated with a layer containing at least 3 atom percent of silicon.

The term “atom percent of silicon” refers to the percentage of silicon atoms on the surface of the pigment relative to the total number of atoms on the surface of the pigment (i.e., carbon, nitrogen, oxygen, sodium, aluminum, silicon, phosphorus, chlorine, calcium, and iron) as determined by ESCA.

As used herein, the term “pigment” means an insoluble colorant. A wide variety of organic and inorganic pigments alone or in combination may be used in the present invention. As used herein, the term “pigment lake” or “lake” refers to a pigment manufactured by precipitating a dye with an inert binder, usually a metallic salt.

As used herein, the term “coated pigment”, also referred to herein as “CP” refers to a pigment coated with a layer containing at least 3 atom percent of silicon, as determined by ESCA. The pigment may be coated with a layer of silica (i.e., silicon dioxide) and/or a silane coupling reagent. The pigment may be partially or completely coated with the silicon-containing layer, provided that the coated pigment contains at least 3 atom percent of silicon on its surface.

The term “ESCA” means electron spectroscopy for chemical analysis, also known as X-ray photoelectron spectroscopy (Practical Surface Analysis, Vol. 1, D. Briggs and M. P. Seah, eds.; John Wiley and Sons, New York, 1983).

As used herein, “S” means molecular spacer. The molecular spacer may be an organic spacer or a peptide spacer, or a combination thereof, as described herein.

As used herein, the term “peptide linker” refers to a peptide spacer used to link together two or more body surface-binding peptides (“fingers”). In one embodiment, the peptide linker is 1 to 60 amino acids in length, preferably 3 to 50 amino acids in length. Examples of peptide linkers are provided as SEQ ID NOs:219-223.

The term “hair” as used herein refers to human hair, eyebrows, and eyelashes.

As used herein, the term “hair-binding peptide” (HBP) refers to a peptide that binds with high affinity to hair. Examples of hair-binding peptides (referred to herein as “fingers”) are provided in Table A. The hair-binding fingers may be linked together to form hair-binding domains (referred to herein as “hands”).

As used herein, the terms “hair hand” and “hair-binding domain” refer to a single chain peptide comprising at least two hair-binding peptides linked together by an optional molecular spacer, wherein the inclusion of a molecular spacer is preferred. In one embodiment, the molecular spacer is a peptide linker.

The term “skin” as used herein refers to human skin, or substitutes for human skin, such as pig skin, VITRO-SKIN® (Innovative Measurement Solutions Inc., Milford, Conn.) and EPIDERM™ (MatTek Corporation, Ashland, Mass.). Skin, as used herein, refers to a body surface generally comprising a layer of epithelial cells and may additionally comprise a layer of endothelial cells.

As used herein, the term “skin-binding peptide” (SBP) refers to peptides that bind with high affinity to skin. Examples of skin-binding peptides (“fingers”) are provided in Table A. The skin-binding fingers may be linked together to form skin-binding domains (“hands”).

As used herein, the terms “skin hand” and “skin-binding domain” refer to a single chain peptide comprising at least two skin-binding peptides linked together by an optional molecular spacer, wherein the inclusion of a molecular spacer is preferred. In one embodiment, the molecular spacer is a peptide linker.

As used herein, the term “nails” refers to human fingernails and toenails.

As used herein, the term “nail-binding peptide” (NBP) refers to peptide sequences that bind with high affinity to nails. Examples of nail-binding peptides (“fingers”) are provided in Table A. The nail-binding fingers may be linked together to form nail-binding domains (“hands”).

As used herein, the terms “nail hand” and “nail-binding domain” refer to a single chain peptide comprising at least two nail-binding peptides linked together by an optional molecular spacer, wherein the inclusion of a molecular spacer is preferred. In one embodiment, the molecular spacer is a peptide linker.

As used herein, the term “oral cavity surface-binding peptide” refers to peptides that bind with high affinity to teeth, gums, cheeks, tongue, or other surfaces in the oral cavity. In one embodiment, the oral cavity surface-binding peptide is a tooth-binding peptide.

As used herein, the term “tooth-binding peptide” (TBP) refers to a peptide that binds with high affinity to tooth enamel or tooth pellicle. Examples of tooth-binding peptides (“fingers”) are provided in Table A. The tooth-binding fingers may be linked together to form tooth-binding domains (“hands”).

As used herein, the terms “tooth hand” and “tooth-binding domain” refer to a single chain peptide comprising at least two tooth-binding peptides linked together by an optional molecular spacer, wherein the inclusion of a molecular spacer is preferred. In one embodiment, the molecular spacer is a peptide linker.

The term “tooth surface” refers to a surface comprised of tooth enamel (typically exposed after professional cleaning or polishing) or tooth pellicle (an acquired surface comprising salivary glycoproteins). Hydroxyapatite can be coated with salivary glycoproteins to mimic a natural tooth pellicle surface (tooth enamel is predominantly comprised of hydroxyapatite).

As used herein, the terms “pellicle” and “tooth pellicle” refer to the thin film (typically ranging from about 1 μm to about 200 μm thick) derived from salivary glycoproteins which forms over the surface of the tooth crown. Daily tooth brushing tends to only remove a portion of the pellicle surface while abrasive tooth cleaning and/or polishing (typically by a dental professional) will exposure more of the tooth enamel surface.

As used herein, the terms “enamel” and “tooth enamel” refer to the highly mineralized tissue which forms the outer layer of the tooth. The enamel layer is composed primarily of crystalline calcium phosphate (i.e. hydroxyapatite) along with water and some organic material. In one embodiment, the tooth surface is selected from the group consisting of tooth enamel and tooth pellicle.

As used herein, the terms “binding affinity” and “affinity” refer to the strength of the interaction of a binding peptide (e.g. body surface-binding peptides, body surface-binding domains, and peptide-based coloring reagents) with a body surface. The binding affinity may be reported in terms of the MB₅₀ value as determined in an ELISA-based binding assay or as a K_{D} (equilibrium dissociation constant) value, which may be deduced using surface plasmon resonance (SPR).

As used herein, the term “MB₅₀” refers to the concentration of the binding peptide that gives a signal that is 50% of the maximum signal obtained in an ELISA-based binding assay (see Example 9 of U.S. Published Patent Application No. 2005-0226839; hereby incorporated by reference). The MB₅₀ provides an indication of the strength of the binding affinity of the binding peptide with a body surface. The lower the value of MB₅₀, the stronger the interaction of the binding peptide with its corresponding body surface.

As used herein, the term “strong affinity” refers to a binding affinity, as measured as an MB₅₀ or K_{D} value, of 10⁻⁴ M or less, preferably less than 10⁻⁵ M, more preferably less than 10⁻⁶ M, more preferably less than 10⁻⁷ M, even more preferably less than 10⁻⁸ M, and most preferably less than 10⁻⁹ M.

The terms “coupling” and “coupled” as used herein refer to a covalent bond.

The term “covalent bond” as used herein refers to a type of chemical bonding that is characterized by the sharing of pairs of electrons between atoms.

The term “amino acid” refers to the basic chemical structural unit of a protein or polypeptide.

The term “gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

The term “promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from a nucleic acid fragment. Expression may also refer to translation of mRNA into a polypeptide.

The term “host cell” refers to a cell which has been transformed or transfected, or is capable of transformation or transfection by an exogenous polynucleotide sequence.

The terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded

DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phages continuously. It is a single-stranded DNA phage.

The term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, NY (2001); and by Silhavy, T. J., Bennan, M. L. and Enquist, L. W., Experiments with Gene Fusions, Cold Spring Harbor Laboratory Cold Press Spring Harbor, NY (1984); and by Ausubel, F. M. et. al., Short Protocols in Molecular Biology, 5^{th} Ed. Current Protocols and John Wiley and Sons, Inc., N.Y., 2002.

### Pigments

Pigments for coloring hair, skin, and other body surfaces are well known in the art (see for example Green et al. (WO 0107009), CFTA International Color Handbook, 2^{nd} ed., Micelle Press, England (1992) and Cosmetic Handbook, US Food and Drug Administration, FDA/IAS Booklet (1992)), and are available commercially from various sources (for example Bayer, Pittsburgh, Pa.; Ciba-Geigy, Tarrytown, N.Y.; ICI, Bridgewater, N.J.; Sandoz, Vienna, Austria; BASF, Mount Olive, N.J.; and Hoechst, Frankfurt, Germany). Exemplary pigments include, but are not limited to, D&C Red No. 36, D&C Red No. 30, D&C Orange No. 17, Green 3 Lake, Ext. Yellow 7 Lake, Orange 4 Lake, and Red 28 Lake; the calcium lakes of D&C Red Nos. 7, 11, 31 and 34, the barium lake of D&C Red No. 12, the strontium lake D&C Red No. 13, the aluminum lakes of FD&C Yellow No. 5, of FD&C Yellow No. 6, of FD&C No. 40, of D&C Red Nos. 21, 22, 27, and 28, of FD&C Blue No. 1, of D&C Orange No. 5, of D&C Yellow No. 10, the zirconium lake of D&C Red No. 33; Cromophthal® Yellow 131 AK (Ciba Specialty Chemicals), Sunfast® Magenta 122 (Sun Chemical) and Sunfast® Blue 15:3 (Sun Chemical), iron oxides, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide, black titanium dioxide, titanium dioxide nanoparticles, zinc oxide, barium oxide, ultramarine blue, bismuth citrate, and white minerals such as hydroxyapatite, and Zircon (zirconium silicate), and carbon black particles.

In one embodiment, the pigment is a metallic oxide, such as iron oxide, titanium dioxide, black titanium dioxide, titanium dioxide nanoparticles, zinc oxide, or barium oxide. In another embodiment, the pigment is iron oxide.

Metallic and semiconductor nanoparticles may also be used as hair coloring agents due to their strong emission of light (Vic et al., U.S. Patent Application Publication No. 2004/0010864). The metallic nanoparticles include, but are not limited to, particles of gold, silver, platinum, palladium, iridium, rhodium, osmium, iron, copper, cobalt, and alloys composed of these metals. An “alloy” is herein defined as a homogeneous mixture of two or more metals. The “semiconductor nanoparticles” include, but are not limited to, particles of cadmium selenide, cadmium sulfide, silver sulfide, cadmium sulfide, zinc oxide, zinc sulfide, zinc selenide, lead sulfide, gallium arsenide, silicon, tin oxide, iron oxide, and indium phosphide. The nanoparticles are stabilized and made water-soluble by the use of a suitable organic coating or monolayer. As used herein, monolayer-protected nanoparticles are one type of stabilized nanoparticle.

Methods for the preparation of stabilized, water-soluble metal and semiconductor nanoparticles are known in the art, and suitable examples are described by Huang et al. in copending and commonly owned U.S. Patent Application Publication No. 2004/0115345, which is incorporated herein by reference. The color of the nanoparticles depends on the size of the particles. Therefore, by controlling the size of the nanoparticles, different colors may be obtained.

Suitable pigments for use herein have a particle diameter of less than 500 nm, preferably between 70 nm and 400 nm.

### Coated Pigments

For use in the invention, the pigment is coated such that its surface contains at least 3 atom percent of silicon. The amount of silicon present on the surface of the coated pigment is determined using ESCA, as described in the Examples herein. In some embodiments, the coated pigment has less than about 40 atom percent of metal atoms on the surface.

In one embodiment, the pigment is coated with silica (i.e., silicon dioxide). The pigment can be coated with a surface layer of silica using methods known in the art. For example, a silica-coated pigment may be prepared by reacting a pigment with an alkali silicate in the presence of a mineral acid while maintaining the pH between 7 to 11 (Jacobson, U.S. Pat. No. 5,340,393 and references therein). This method, which is applicable to a broad range of pigment particles, is described in detail in Example 1 herein. Silica-coated pigments may also be prepared using well known sol-gel chemistry, in which a silica sol-gel coating is formed on the surface of the pigment particles by the hydrolysis and condensation of an inorganic metal alkoxide, such as tetraethylorthoxisilicate. Organic pigment particles may be coated with silica using the sol-gel process described by Yuan et al. (Journal of Sol-Gel Science and Technology 36:265-274, 2005). In that method, the surface of an organic pigment is first modified by poly(sodium 4-styrenesulfonate) and poly(diallydimethylammonium chloride), then coated by silica using a sol-gel process with tetraethylorthoxisilicate. Additionally, silica-coated pigments are available commercially from companies such as Presperse, Inc. (Somerset, N.J.), Color Techniques, Inc. (South Plainfield N.J.), and Kobo Products, Inc. (South Plainfield N.J.).

The silica-coated pigment may then be reacted with a silane coupling reagent to introduce reactive groups on the surface of the pigment that are capable of forming covalent bonds with a body surface-binding peptide. Suitable examples of silane coupling reagents include, but are not limited to, isocyanatopropylsilane, mercaptopropylsilane, aminopropylsilane, 3-chloropropyltrimethoxysilane, 3-chloropropyltriethoxysilane, 3-chloropropylmethyldimethoxysilane, 3-chloropropylmethyldiethoxysilane, vinyltrimethoxysilane, methacryloxypropyltrimethoxysilane, vinyltriethoxysilane, and combinations thereof. In one embodiment, the silane coupling reagent is isocyanatopropylsilane, mercaptopropylsilane, or aminopropylsilane. In another embodiment, the silane coupling reagent is isocyanatopropylsilane. The isocyanatopropylsilane has isocyanate groups which will form covalent bonds with amine and hydroxyl groups on the body surface-binding peptide. The mercaptopropylsilane has sulfhydryl groups which will form disulfide bonds with sulhydryl groups in cysteine residues on the peptide. Combinations of these silane coupling reagents may be used to introduce more than one type of reactive group on the surface of the coated pigment.

Pigments having surface hydroxyl groups (e.g., metal oxides) may be coated such that the surface contains at least 3 atom percent of silicon by reacting the pigment with a silane coupling reagent, as described above for the silica-coated pigments. In this embodiment, a silica coating is not required. The silane reagent provides both the silicon on the surface and the reactive group(s) for covalent bonding of the pigment to the body surface-binding peptide.

### Body Surfaces

Body surfaces are any surface on the human body that will serve as a substrate for a binding peptide. In one embodiment, the body surfaces are selected from the group consisting of hair, skin, nails, teeth, gums, and other tissues of the oral cavity. In many cases the body surfaces of the invention will be exposed to air, however in some instances, the oral cavity for example, the surfaces will be internal. Accordingly, body surfaces may include layers of both epithelial and well as endothelial cells.

Samples of body surfaces for use in the identification of body surface-binding peptides are available from a variety of sources. For example, human hair samples are available commercially, for example from International Hair Importers and Products (Bellerose, NY), in different colors, such as brown, black, red, and blond, and in various types, such as African-American, Caucasian, and Asian. Additionally, the hair samples may be treated for example using hydrogen peroxide to obtain bleached hair. Human skin samples may be obtained from cadavers or in vitro human skin cultures. Additionally, pig skin, available from butcher shops and supermarkets, VITRO-SKIN®, available from IMS Inc. (Milford, Conn.), and EPIDERM™, available from MatTek Corp. (Ashland, Mass.), are good substitutes for human skin. Human fingernails and toenails may be obtained from volunteers. Extracted mammalian teeth, such as bovine and/or human teeth are commercially available. Extracted human teeth may also be obtained from dental offices. Additionally, hydroxyapatite, available in many forms, for example, from Berkeley Advanced Biomaterials, Inc. (San Leandro, Calif.), may be used (once coated with salivary glycoproteins to form an acquired pellicle) as a model for studying teeth-binding peptides (see copending and commonly owned U.S. Patent Application Publication No. 2008/0280810).

### Body Surface-Binding Peptides

Body surface-binding peptides, as defined herein, are peptide sequences that specifically bind with high affinity to a specific body surface including, but not limited to hair, nails, skin, and teeth. In one embodiment, the body surface-binding peptide is selected from the group consisting of hair-binding peptides, skin-binding peptides, nail-binding peptides, and tooth-binding peptides.

The body surface-binding peptides are from about 7 amino acids to about 60 amino acids in length, more preferably, from about 7 amino acids to about 35 amino acids in length, most preferably from about 7 to about 20 amino acids in length. In a preferred embodiment, the body surface-binding peptides are combinatorially-generated peptides.

Phage display has been used to identify various body surface-binding peptides. For example, peptides having an affinity for a body surface have been described in U.S. Pat. Nos. 7,220,405 and 7,285,264; U.S. Patent Application Publications Nos. US 2005/0226839, US 2005/0249682, US 2007/0065387, US 2007/0067924, US 2007/0196305, US 2007/0110686, US 2006/0073111, US 2006/0199206, US 2008/0280810, and US 2008/0175798; and PCT Patent Application Publication No. WO 2004/048399. Examples of various body surface-binding peptides are provided in Table A.

TABLE A
Examples of Body Surface-Binding Peptides

| | SEQ ID | |
|---|---|---|
| Body Surface | NO: | Reference |
| Hair | 1-42, | U.S. Pat. No. 7,220,405 |
| | 90-109 | |
| Hair | 43-65 | WO2004048399 |
| Hair | 66 | US 2007/0065387 |
| Hair | 67, 68, | US 2007/0067924 |
| | 75 | |
| Hair | 69-74 | US 2008/0280810 |
| Hair | 76-79, | US 2008/0175798 |
| | 81-88 | |
| Hair | 80, | US 2007/0196305 |
| | 110-114, | |
| | 118, | |
| Hair | 89 | U.S. Pat. No. 7,285,264 |
| Hair | 115-116, | US 2006/0073111 |
| | 119-122 | |
| Hair | 117 | US 2007/0067924 |
| | | U.S. Pat. No. 7,285,264 |
| Hair and skin | 123 | US 2007/0065387 |
| | | US 2007/0110686 |
| | | US 2007/0067924 |
| Hair and skin | 124 | US 2007/0065387 |
| | | US 2007/0110686 |
| Hair and skin | 125 | US 2007/0065387 |
| | | US 2007/0110686 |
| Hair and skin | 126 | US 2007/0065387 |
| | | US 2007/0110686 |
| Hair and skin | 127 | US 2007/0065387 |
| | | US 2007/0110686 |
| Skin | 128 | US 2008/0280810 |
| | | US 2005/0249682 |
| Skin | 129-132 | US 2007/0110686 |
| Skin | 133 | US 2008/0280810 |
| | | US 2005/0249682 |
| Skin | 134 | US 2008/0280810 |
| | | US 2005/0249682 |
| | | WO2004048399 |
| Skin | 135 | US 2008/0280810 |
| | | US 2005/0249682 |
| | | WO2004048399 |
| Skin | 136 | US 2008/0280810 |
| | | US 2005/0249682 |
| | | WO2004048399 |
| Skin | 137 | US 2005/0249682 |
| | | WO2004048399 |
| Skin | 138 | US 2005/0249682 |
| | | WO2004048399 |
| Skin | 139 | US 2005/0249682 |
| | | WO2004048399 |
| Skin | 140-157 | WO2004048399 |
| Skin | 158-175 | US 2008/0280810 |
| | | US 2006/0199206 |
| Fingernail | 176 | US 2005/0226839 |
| | | U.S. Pat. No. 7,220,405 |
| Fingernail and | 177 | US 2005/0226839 |
| Hair | | U.S. Pat. No. 7,220,405 |
| Tooth (pellicle) | 178-197 | US 2008/0280810 |
| Tooth | 198-217 | US 2008/0280810 |
| (enamel) | | |

Additional body surface-binding peptides may be identified using phage display as described in the references cited above.

Alternatively, hair-binding and skin-binding peptide sequences may be generated empirically by designing peptides that comprise positively charged amino acids, which can bind to hair and skin via electrostatic interaction, as described by Rothe et al. (WO 2004/000257). The empirically generated hair and skin-binding peptides have between about 4 amino acids to about 50 amino acids, preferably from about 4 to about 25 amino acids, and comprise at least about 40 mole % positively charged amino acids, such as lysine, arginine, and histidine. Peptide sequences containing tripeptide motifs such as HRK, RHK, HKR, RKH, KRH, KHR, HKX, KRX, RKX, HRX, KHX and RHX are most preferred where X can be any natural amino acid but is most preferably selected from neutral side chain amino acids such as glycine, alanine, proline, leucine, isoleucine, valine and phenylalanine. In addition, it should be understood that the peptide sequences must meet other functional requirements in the end use including solubility, viscosity and compatibility with other components in a formulated product and will therefore vary according to the needs of the application. In some cases the peptide may contain up to 60 mole % of amino acids not comprising histidine, lysine or arginine. Suitable empirically generated hair-binding and skin peptides include, but are not limited to, SEQ ID NOs:123-127.

It may also be desirable to link body surface-binding peptide sequences together to form body surface-binding domains (“binding hand”) in order to enhance the interaction between the peptide-based coloring reagent and the body surface, as described by Huang et al. (U.S. Patent Application Publication No.2005/0050656). Either multiple copies of the same body surface-binding peptide or a combination of different body surface-binding peptides may be used. The body surface-binding peptides may be linked directly or through a spacer. Any known peptide or protein conjugation chemistry may be used to link the body surface-binding peptides together to form the body surface-binding domains. Conjugation chemistries are well-known in the art (see for example, G. T. Hermanson, Bioconjugate Techniques, 2^{nd} Ed., Academic Press, New York (2008)). Suitable coupling agents include, but are not limited to, carbodiimide coupling agents, diacid chlorides, diisocyanates and other difunctional coupling reagents that are reactive toward terminal amine and/or carboxylic acid groups on the peptides. Alternatively, body surface-binding domains may be prepared using recombinant DNA and molecular cloning techniques, described below.

It may also be desirable to link the body surface-binding peptides together via a molecular spacer to form body surface-binding domains. The molecular spacer serves to separate the body surface-binding peptide sequences to ensure that the binding affinity of the individual peptides is not adversely affected by the coupling. The molecular spacer may also provide other desirable properties such as hydrophilicity, hydrophobicity, or a means for cleaving the peptide sequences to facilitate removal of the pigment. The molecular spacer may be an organic spacer or a peptide spacer. The organic spacer may be any of a variety of molecules, such as alkyl chains, phenyl compounds, ethylene glycol, amides, esters and the like. Preferred organic spacers are hydrophilic and have a chain length from 1 to about 100 atoms, more preferably, from 2 to about 30 atoms. Examples of preferred organic spacers include, but are not limited to ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl chains, and ethyl, propyl, hexyl, steryl, cetyl, and palmitoyl alkyl chains. The spacer may be covalently attached to the body surface-binding peptide sequences using any of the coupling chemistries described above.

The peptide spacer used to link together body surface-binding peptides, also referred to herein as a peptide linker, is a peptide, which may comprise any amino acid and mixtures thereof. The preferred peptide spacers comprise the amino acids proline, lysine, glycine, alanine, cysteine, and serine, and mixtures thereof. In addition, the peptide spacer may contain a specific enzyme cleavage site, such as the protease Caspase 3 site, given by SEQ ID NO:218, which allows for the enzymatic removal of the pigment from the body surface. The peptide spacer may be from 1 to about 60 amino acids, preferably from 3 to about 50 amino acids. Examples of peptide spacers include, but are not limited to, the sequences given by SEQ ID NOs:219-223). These peptide spacers may be linked to the binding peptide sequence by any method know in the art. For example, the entire body surface-binding domain including the peptide spacer(s) may be prepared using the standard peptide synthesis methods described below. In addition, the body surface-binding peptides and peptide spacer(s) may be combined using carbodiimide coupling agents (see for example, Hermanson, Bioconjugate Techniques, Academic Press, New York (1996)), diacid chlorides, diisocyanates and other difunctional coupling reagents that are reactive to terminal amine and/or carboxylic acid terminal groups on the peptides. Alternatively, the entire body surface-binding domain may be prepared using the recombinant DNA and molecular cloning techniques described below. The molecular spacer may also be a combination of a peptide spacer and an organic spacer molecule, which may be prepared using the methods described above.

Examples of body surface-binding domains (i.e., hair-binding domains) comprising peptide spacer(s) are given as SEQ ID NOs:224-229.

### Production of Binding Peptides

Suitable body surface-binding peptides may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, body surface-binding peptides may be prepared using recombinant DNA and molecular cloning techniques. Genes encoding the peptides may be produced in heterologous host cells, particularly in the cells of microbial hosts.

Preferred heterologous host cells for expression of the body surface-binding peptides are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of suitable host strains include, but are not limited to, fungal or yeast species such as Aspergillus, Trichoderma, Saccharomyces, Pichia, Candida, Yarrowia, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella.

A variety of expression systems can be used to produce body surface-binding peptides. Such vectors include, but are not limited to, chromosomal, episomal and virus-derived vectors, e.g., vectors derived from bacterial plasmids, from bacteriophage, from transposons, from insertion elements, from yeast episomes, from viruses such as baculoviruses, retroviruses and vectors derived from combinations thereof such as those derived from plasmid and bacteriophage genetic elements, such as cosmids and phagemids. The expression system constructs may contain regulatory regions that regulate as well as engender expression. In general, any system or vector suitable to maintain, propagate or express polynucleotide or polypeptide in a host cell may be used for expression in this regard. Microbial expression systems and expression vectors contain regulatory sequences that direct high level expression of foreign proteins relative to the growth of the host cell. Regulatory sequences are well known to those skilled in the art and examples include, but are not limited to, those which cause the expression of a gene to be turned on or off in response to a chemical or physical stimulus, including the presence of regulatory elements in the vector, for example, enhancer sequences. Any of these may be used to construct chimeric genes for production of body-surface-binding peptides. These chimeric genes could then be introduced into appropriate microorganisms via transformation to provide high level expression of the peptides.

Vectors or cassettes useful for the transformation of suitable host cells are well known in the art. Typically the vector or cassette contains sequences directing transcription and translation of the relevant gene, one or more selectable markers, and sequences allowing autonomous replication or chromosomal integration. Suitable vectors comprise a region 5′ of the gene, which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Selectable marker genes provide a phenotypic trait for selection of the transformed host cells such as tetracycline or ampicillin resistance in E. coli.

Initiation control regions or promoters which are useful to drive expression of the chimeric gene in the desired host cell are numerous and familiar to those skilled in the art. Virtually any promoter capable of driving the gene is suitable for producing body surface-binding peptides including, but not limited to: CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, araB, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

The vector containing the appropriate DNA sequence, as well as an appropriate promoter or control sequence, may be employed to transform an appropriate host to permit the host to express a body surface-binding peptide. Cell-free translation systems can also be employed to produce such peptides using RNAs derived from the DNA constructs of the present invention. Optionally it may be desired to produce the instant gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the production host. Methods for choosing appropriate signal sequences are well known in the art (see for example EP 546049 and WO 9324631). The secretion signal DNA or facilitator may be located between the expression-controlling DNA and the instant gene or gene fragment, and in the same reading frame with the latter.

### Peptide-Based Coloring Reagents

The peptide-based coloring reagents of the invention comprise at least one body surface-binding peptide covalently attached to the surface of a coated pigment. The peptide-based coloring reagents of the invention will include body-surface binding peptides that are comprised of one or more peptide fingers or hands bound to a particle, either directly or via a spacer. The peptide-based coloring reagents may be prepared in various ways. For example, a silica-coated pigment that has been treated with a silane coupling reagent having reactive groups (e.g., isocyanate or sulfhydryl) that will form a covalent bond with a body surface-binding peptide may be reacted directly with the peptide to form a peptide-based coloring reagent. Similarly, a pigment without a silica coating that has been treated with a silane coupling reagent having reactive groups (e.g., isocyanate or sulfhydryl) that will form a covalent bond with a body surface-binding peptide may also be reacted directly with the peptide to form a peptide-based coloring reagent.

Additionally, silica-coated or uncoated pigments that have been treated with a silane coupling agent may be covalently coupled via a molecular spacer. The molecular spacer serves to separate the body surface-binding peptide from the pigment particle to ensure that the binding affinity of the body surface-binding peptide is not adversely affected by the pigment. The molecular spacer may be an organic spacer or a peptide spacer, as described above. In order to facilitate incorporation of an organic spacer, a bifunctional cross-linking agent that contains a spacer and reactive groups at both ends for coupling the body surface-binding peptide to the coated pigment may be used. For example, a coated pigment having a primary amine group on the surface may be covalently attached to the body surface-binding peptide using bifunctional crosslinking agents such as dialdehydes (e.g., glutaraldehyde), bis N-hydroxysuccinimide esters (e.g., ethylene glycol-bis(succinic acid N-hydroxysuccinimide ester), disuccinimidyl glutarate, disuccinimidyl suberate, and ethylene glycol-bis(succinimidylsuccinate)), diisocyanates (e.g., hexamethylenediisocyanate), bis oxiranes (e.g., 1,4 butanediyl diglycidyl ether), and the like. Heterobifunctional cross-linking agents, which contain a different reactive group at each end, may also be used. An example of a useful heterobifunctional crosslinking agent is (succinimidyl-[(N-maleimidopropionamido)-diethyleneglycol] ester), available from Pierce Biotechnology (Rockford, Ill.), which has a succinimidyl ester group for covalent attachment to amine groups on the coated pigment and a maleimide group for covalent attachment to cysteine residues on the body surface-binding peptide. Additionally, a peptide spacer comprising lysine or cysteine residues may be added to the body surface-binding peptide sequence to facilitate covalent attachment to the coated pigment.

The peptide-based coloring reagents of the invention may also be prepared by reacting a silane coupling agent with a body surface-binding peptide to form a silanized peptide, which is then reacted with a silica-coated pigment. Any of the conjugation chemistries described above may be used to covalently attach the silane coupling reagent to the body surface-binding peptide.

Therefore, in one embodiment the peptide-based coloring reagent is represented by the general structure:

(BSBP)ₙ—CP, or

[(BSBP)ₘ—S]ₙ—CP

wherein: BSBP is a body surface-binding peptide; CP is a coated pigment containing at least 3 atom percent of silicon on its surface, as determined by ESCA; S is a molecular spacer; BSBP is covalently bound to the surface of CP in the first structure and S is covalently bound to the surface of CP in the second structure; m ranges from 1 to about 50; and n ranges from 1 to about 100,000.

In another embodiment, the body surface-binding peptide is a hair-binding peptide and the peptide-based coloring reagent is represented by the general structure:

(HPB)ₙ—CP, or

[(HBP)ₘ—S]ₙ—CP

wherein: HBP is a hair-binding peptide; CP is a coated pigment containing at least 3 atom percent of silicon on its surface, as determined by ESCA; S is a molecular spacer; HBP is covalently bound to the surface of CP in the first structure and S is covalently bound to the surface of CP in the second structure; m ranges from 1 to about 50; and n ranges from 1 to about 100,000.

In another embodiment, the body surface-binding peptide is a skin-binding peptide and the peptide-based coloring reagent is represented by the general structure:

(SBP)ₙ—CP, or

[(SBP)ₘ—S]ₙ—CP

wherein: SBP is a hair-binding peptide; CP is a coated pigment containing at least 3 atom percent of silicon on its surface, as determined by ESCA; S is a molecular spacer; SBP is covalently bound to the surface of CP in the first structure and S is covalently bound to the surface of CP in the second structure; m ranges from 1 to about 50; and n ranges from 1 to about 100,000.

In another embodiment, the body surface-binding peptide is a nail-binding peptide and the peptide-based coloring reagent is represented by the general structure:

(NBP)ₙ—CP, or

[(NBP)ₘ—S]ₙ—CP

wherein: NBP is a hair-binding peptide; CP is a coated pigment containing at least 3 atom percent of silicon on its surface, as determined by ESCA; S is a molecular spacer; NBP is covalently bound to the surface of CP in the first structure and S is covalently bound to the surface of CP in the second structure; m ranges from 1 to about 50; and n ranges from 1 to about 100,000.

In another embodiment, the body surface-binding peptide is a tooth-binding peptide and the peptide-based coloring reagent is represented by the general structure:

(TBP)ₙ—CP, or

[(TBP)ₘ—S]ₙ—CP

wherein: TBP is a hair-binding peptide; CP is a coated pigment containing at least 3 atom percent of silicon on its surface, as determined by ESCA; S is a molecular spacer; TBP is covalently bound to the surface of CP in the first structure and S is covalently bound to the surface of CP in the second structure; m ranges from 1 to about 50; and n ranges from 1 to about 100,000.

It should be understood that as used herein BSBP, HBP, SBP, NBP, and TBP are generic designations and are not meant to refer to a single body surface-binding peptide, hair-binding peptide, skin-binding peptide, nail-binding peptide, or tooth-binding sequence, respectively. Where m or n as used above, is greater than 1, it is well within the scope of the invention to provide for the situation where a series of body surface-binding peptides of different sequences may form a part of the composition. Additionally, S is a generic term and is not meant to refer to a single molecular spacer. Where n, as used above, is greater than 1, it is well within the scope of the invention to provide for the situation where a series of different spacers may form a part of the composition. In a preferred embodiment, the peptide-based coloring reagent is a linear, recombinantly produced peptide comprising at least one body surface-binding peptide, and optionally one or more peptide spacers, covalently attached to a coated pigment.

### Personal Care Compositions

The peptide-based coloring reagents of the invention may be used in personal care compositions to color body surfaces, such as hair, skin, nails, and teeth. The body surface-binding peptide of the peptide-based coloring reagent has an affinity for the body surface, thereby attaching the pigment to the body surface. Personal care compositions include, but are not limited to, hair care compositions, hair coloring compositions, skin care compositions, cosmetic compositions, nail polish compositions, and oral care compositions.

Hair Care Compositions

In one embodiment, the peptide-based coloring reagent is a component of a hair care composition and the peptide-based coloring reagent comprises at least one hair-binding peptide. Hair care compositions are herein defined as compositions for the treatment of hair including, but not limited to, shampoos, conditioners, rinses, lotions, aerosols, gels, and mousses. An effective amount of the peptide-based coloring reagent for use in hair care compositions is a concentration of about 0.01% to about 10%, preferably about 0.01% to about 5% by weight relative to the total weight of the composition. This proportion may vary as a function of the type of hair care composition. Additionally, a mixture of different peptide-based coloring reagents comprising different pigments may be used in the composition. Suitable mixtures of peptide-based coloring reagents may be determined by one skilled in the art using routine experimentation. If a mixture of peptide-based coloring reagents is used in the composition, the total concentration of the reagents is about 0.01% to about 10% by weight relative to the total weight of the composition.

The composition may further comprise a cosmetically acceptable medium for hair care compositions, examples of which are described by Philippe et al. in U.S. Pat. No. 6,280,747, and by Omura et al. in U.S. Pat. No. 6,139,851 and Cannell et al. in U.S. Pat. No. 6,013,250, all of which are incorporated herein by reference. For example, these hair care compositions can be aqueous, alcoholic or aqueous-alcoholic solutions, the alcohol preferably being ethanol or isopropanol, in a proportion of from about 1 to about 75% by weight relative to the total weight for the aqueous-alcoholic solutions. Additionally, the hair care compositions may contain one or more conventional cosmetic or dermatological additives or adjuvants including, but not limited to, antioxidants, preserving agents, fillers, surfactants, UVA and/or UVB sunscreens, fragrances, thickeners, wetting agents and anionic, nonionic or amphoteric polymers, and dyes.

Hair Coloring Compositions

In another embodiment, the peptide-based coloring reagent is a component of a hair coloring composition and the peptide-based coloring reagent comprises at least one hair binding peptide. Hair coloring compositions are herein defined as compositions for the coloring or dyeing of hair.

An effective amount of a peptide-based coloring reagent for use in a hair coloring composition is herein defined as a proportion of from about 0.01% to about 20% by weight relative to the total weight of the composition. Additionally, a mixture of different peptide-based reagents comprising different pigments may be used in the composition. Suitable mixtures of peptide-based coloring reagents may be determined by one skilled in the art using routine experimentation. If a mixture of peptide-based coloring reagents is used in the composition, the total concentration of the reagents is about 0.01% to about 20% by weight relative to the total weight of the composition.

Components of a cosmetically acceptable medium for hair coloring compositions are described by Dias et al., in U.S. Pat. No. 6,398,821 and by Deutz et al., in U.S. Pat. No. 6,129,770, both of which are incorporated herein by reference. For example, hair coloring compositions may contain sequestrants, stabilizers, thickeners, buffers, carriers, surfactants, solvents, antioxidants, polymers, and conditioners.

Skin Care Compositions

In another embodiment, the peptide-based coloring reagent is a component of a skin care composition and the peptide-based coloring reagent comprises at least one skin-binding peptide. Skin care compositions are herein defined as compositions for the treatment of skin including, but not limited to, skin care, skin cleansing, make-up, and anti-wrinkle products. An effective amount of the peptide-based coloring reagent for use in a skin care composition is a concentration of about 0.01% to about 10%, preferably about 0.01% to about 5% by weight relative to the total weight of the composition. This proportion may vary as a function of the type of skin care composition. Additionally, a mixture of different peptide-based coloring reagents comprising different pigments may be used in the composition. Suitable mixtures of peptide-based coloring reagents may be determined by one skilled in the art using routine experimentation. If a mixture of peptide-based coloring reagents is used in the composition, the total concentration of the reagents is about 0.01% to about 10% by weight relative to the total weight of the composition.

The composition may further comprise a cosmetically acceptable medium for skin care compositions, examples of which are described by Philippe et al. supra. For example, the cosmetically acceptable medium may be an anhydrous composition containing a fatty substance in a proportion generally of from about 10 to about 90% by weight relative to the total weight of the composition, where the fatty phase contains at least one liquid, solid or semi-solid fatty substance. The fatty substance includes, but is not limited to, oils, waxes, gums, and so-called pasty fatty substances. Alternatively, the compositions may be in the form of a stable dispersion such as a water-in-oil or oil-in-water emulsion. Additionally, the compositions may contain one or more conventional cosmetic or dermatological additives or adjuvants including, but not limited to, antioxidants, preserving agents, fillers, surfactants, UVA and/or UVB sunscreens, fragrances, thickeners, wetting agents and anionic, nonionic or amphoteric polymers, and dyes.

Skin Coloring Compositions

In another embodiment, the peptide-based coloring reagent is a component of a skin coloring composition and the peptide-based coloring reagent comprises at least one skin-binding peptide.

The skin coloring compositions may be any cosmetic or make-up product, including but not limited to foundations, blushes, lipsticks, lip liners, lip glosses, eyeshadows and eyeliners. These may be anhydrous make-up products comprising a cosmetically acceptable medium which contains a fatty substance, or they may be in the form of a stable dispersion such as a water-in-oil or oil-in-water emulsion, as described above. In these compositions, an effective amount of the peptide-based coloring reagent is generally from about 0.01% to about 40% by weight relative to the total weight of the composition. Additionally, a mixture of different peptide-based coloring comprising different pigments may be used in the composition. Suitable mixtures of peptide-based coloring reagents may be determined by one skilled in the art using routine experimentation. If a mixture of peptide-based coloring reagents is used in the composition, the total concentration of the reagents is about 0.01% to about 40% by weight relative to the total weight of the composition.

Cosmetic Compositions

In another embodiment, the peptide-based coloring reagent is a component of a cosmetic composition and the peptide-based coloring reagent comprises at least one hair binding peptide. Cosmetic compositions, as defined herein, are compositions that may be applied to the eyelashes or eyebrows including, but not limited to mascaras, and eyebrow pencils.

An effective amount of a peptide-based coloring reagent for use in a cosmetic composition is herein defined as a proportion of from about 0.01% to about 20% by weight relative to the total weight of the composition. Additionally, a mixture of different peptide-based coloring reagents comprising different pigments may be used in the composition. Suitable mixtures of peptide-based coloring reagents may be determined by one skilled in the art using routine experimentation. If a mixture of peptide-based coloring reagents is used in the composition, the total concentration of the reagents is about 0.01% to about 20% by weight relative to the total weight of the composition.

Cosmetic compositions may be anhydrous make-up products comprising a cosmetically acceptable medium which contains a fatty substance in a proportion generally of from about 10 to about 90% by weight relative to the total weight of the composition, where the fatty phase containing at least one liquid, solid or semi-solid fatty substance, as described above. The fatty substance includes, but is not limited to, oils, waxes, gums, and so-called pasty fatty substances. Alternatively, these compositions may be in the form of a stable dispersion such as a water-in-oil or oil-in-water emulsion, as described above.

Nail Polish Compositions

In another embodiment, the peptide-based coloring reagent is a component of a nail polish composition and the peptide-based coloring reagent comprises at least one nail-binding peptide. The nail polish compositions are used for coloring fingernails and toenails.

An effective amount of a peptide-based coloring reagent for use in a nail polish composition is herein defined as a proportion of from about 0.01% to about 20% by weight relative to the total weight of the composition. Additionally, a mixture of different peptide-based coloring reagents comprising different pigments may be used in the composition. Suitable mixtures of peptide-based coloring reagents may be determined by one skilled in the art using routine experimentation. If a mixture of peptide-based coloring reagents is used in the composition, the total concentration of the reagents is about 0.01% to about 20% by weight relative to the total weight of the composition.

Components of a cosmetically acceptable medium for nail polish compositions are described by Philippe et al. supra. The nail polish composition typically contains a solvent and a film forming substance, such as cellulose derivatives, polyvinyl derivatives, acrylic polymers or copolymers, vinyl copolymers and polyester polymers. Additionally, the nail polish may contain a plasticizer, such as tricresyl phosphate, benzyl benzoate, tributyl phosphate, butyl acetyl ricinoleate, triethyl citrate, tributyl acetyl citrate, dibutyl phthalate or camphor.

Oral Care Compositions

In another embodiment, the peptide-based coloring reagent is a component of an oral care composition and the peptide-based coloring reagent comprises at least one tooth-binding peptide. The oral care compositions of the invention are used to whiten teeth; therefore, the peptide-based coloring reagent comprises a white pigment, such as titanium dioxide and titanium dioxide nanoparticles; and white minerals such as hydroxyapatite, and Zircon (zirconium silicate).

The oral care compositions of the invention may be in the form of powder, paste, gel, liquid, ointment, or tablet. Exemplary oral care compositions include, but are not limited to, toothpaste, dental cream, gel or tooth powder, mouth wash, breath freshener, and dental floss. The oral care compositions comprise an effective amount of the peptide-based coloring reagent of the invention in an orally acceptable carrier medium. An effective amount of a peptide-based coloring reagent for use in an oral care composition may vary depending on the type of product. Typically, the effective amount of the peptide-based coloring reagent is a proportion from about 0.01% to about 90% by weight relative to the total weight of the composition. Additionally, a mixture of different peptide-based coloring reagents comprising different pigments may be used in the composition. Suitable mixtures of peptide-based coloring reagents may be determined by one skilled in the art using routine experimentation. If a mixture of peptide-based coloring reagents is used in the composition, the total concentration of the reagents is about 0.001% to about 90% by weight relative to the total weight of the composition.

Components of an orally acceptable carrier medium are described by White et al. in U.S. Pat. No. 6,740,311; Lawler et al. in U.S. Pat. No. 6,706,256; and Fuglsang et al. in U.S. Pat. No. 6,264,925; all of which are incorporated herein by reference. For example, the oral care composition may comprise one or more of the following: abrasives, surfactants, chelating agents, fluoride sources, thickening agents, buffering agents, solvents, humectants, carriers, bulking agents, and oral benefit agents, such as enzymes, anti-plaque agents, anti-staining agents, anti-microbial agents, anti-caries agents, flavoring agents, coolants, and salivating agents.

### Methods for Coloring a Body Surface

The peptide-based coloring reagents of the invention may be used to color body surfaces, such as hair, skin, nails, and teeth. In one embodiment, a personal care composition comprising at least one peptide-based coloring agent is applied to a body surface for a time sufficient for the peptide-based coloring agent to bind to the body surface.

Methods for Coloring Hair

The peptide-based coloring reagents of the invention may be used to attach a pigment to the surface of the hair, thereby coloring the hair. The peptide-based coloring reagent may be applied to the hair from any suitable hair care composition, for example a hair colorant or hair conditioner composition. These hair care compositions are well known in the art and suitable compositions are described above.

In one embodiment, a composition comprising a peptide-based coloring reagent, for example a hair coloring composition, is applied to the hair for a time sufficient for the peptide-based coloring reagent to bind to the hair, typically between about 5 seconds to about 60 minutes. The hair care composition may be rinsed from the hair or left on the hair.

Methods for Coloring Skin

The peptide-based coloring reagents of the invention may be used to attach a pigment to the surface of the skin, thereby coloring the skin.

The peptide-based coloring reagent may be applied to the skin from any suitable skin care composition, for example a skin colorant or skin conditioner composition. These skin care compositions are well known in the art and suitable compositions are described above.

In one embodiment, a composition comprising a peptide-based coloring reagent is applied to the skin for a time sufficient for the peptide-based coloring reagent to bind to the skin, typically between about 5 seconds to about 60 minutes. Optionally, the skin may be rinsed to remove the composition that has not bound to the skin.

Methods for Coloring Nails, Eyebrows, Eyelashes, and Teeth

The methods described above for coloring hair and skin may also be applied to coloring fingernails and toenails, eyebrows, eyelashes, and teeth by applying the appropriate composition, specifically, a nail polish composition, a cosmetic composition, or an oral care composition, comprising at least one peptide-based coloring reagent to the body surface of interest.

### Examples

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “sec” means second(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmol” means micromole(s), “g” means gram(s), “μg” means microgram(s), “mg” means milligram(s), “rpm” means revolutions per minute, “eV” means electron volt(s), “S” means siemens, “mS” means millisiemens, “μS” means microsiemens, “IEP” means isoelectric point”, “MALDI” means matrix assisted, laser desorption ionization.

### General Methods

### Determination of Isoelectric Point

To determine the isoelectric point (IEP) of the silica-coated pigments, a dispersion of the pigment particles containing 2 wt % solids was prepared and placed in a sonication bath for 1 min. The IEP of the resulting dispersion was determined using a Colloidal Dynamics Acoustosizer (Colloidal Dynamics, North Attleboro, Ma.) at a run temperature of 25° C.

### ESCA Analysis of Coated Pigments

ESCA analysis was done using a PHI Model Quantera®SXM instrument (Physical Electronics USA, Chanhassen, Minn.). Monochromatized aluminum K-alpha X-rays were focused on the iron oxide powders, which were pressed into Indium foil, and the kinetic energies of photo-excited core electrons were analyzed by a hemispherical energy analyzer, with pass energy set to 55 eV. Charge compensation in the form of a dual electron and argon ion beam system was used. Data was collected from a 1500×200 μm² area. The exit angle of the photoelectrons detected was 45 degrees. Quantification was based on peak areas calculated after Shirley background subtraction, by multiplication with calculated atomic sensitivity factors corrected for the analyzer transmission function. Atom % concentrations were normalized to 100%.

### Example 1

### Preparation of Silica-Coated Red Iron Oxide Pigment

This Example illustrates the preparation of a silica-coated red iron oxide pigment. A dispersion of the red oxide pigment Unipure red LC 381 was reacted with sodium silicate to prepare the silica-coated red pigment.

A dispersion of the red iron oxide pigment Unipure red LC 381 was prepared as follows. Deionized water (300 g) and 2.0 g of sodium pyrophosphate were added to a 1-L tank on a high speed disperser and stirred to dissolve. Then, 100 g of Unipure red LC 381 pigment (obtained from Sensient Technologies, Milwaukee, Wis.) was added and the mixture was mixed at 8000 rpm for 30 min to give a dispersion of the red iron oxide pigment. The average particle size of the dispersion was measured to be 300 nm with a particle size analyzer using laser diffraction (Mastersizer 2000 Particle Analyzer, Malvern Instruments, West Borough, Mass.).

To prepare the silica-coated red iron oxide pigment, 1454 g of the red iron oxide pigment dispersion (363.5 g of red iron oxide) was charged to a 3-L round bottom flask equipped with a mechanical stirring blade, thermocouple, heating mantle, two addition funnels, pH probe, water condenser and a nitrogen inlet. Deionized water (363.5 g) was added to bring the dispersion to 20% solids. Then, 45.5 g of sodium silicate solution (27% SiO₂, 14% NaOH) in 279.5 g of water was charged to one of the addition funnels and 5.2 g of H₂SO₄ in 319.8 g of deionized water was added to the other. The dispersion was heated to 90° C. and the pH was adjusted to 10.5 with the addition of 37 g of 28% NH₄OH. The sodium silicate solution and the sulfuric acid solution were co-added over a four hour period of time with the reaction mixture at 90° C. The reaction mixture was cooled to 47° C. and brought to pH 7.4 with 3.5 g of sulfuric acid to yield 2472 g of a red dispersion containing silica-coated red iron oxide pigment. The dispersion was de-salted using ultrafiltration as follows. The dispersion and 322 g of rinse water were added to an ultrafiltration tank along with 1.0 L of pH 8.6 deionized water. A total of 8.0 L of deionized water was added and 9.0 L of permeate was removed in 1-L increments. The conductivity of the first liter of permeate was 5.4 mS and of the ninth liter was 470 μS. The dispersion was then put in the high speed disperser for 15 min at 8000 rpm, after which time 1994 g of dispersion was collected that contained 14.85 wt % solids with an average particle size of 527 nm and an IEP of 2.0.

A silica-coated red iron oxide pigment dispersion having an IEP of 5 was prepared from the silica-coated red iron oxide dispersion having an IEP of 2.0 as follows. A 3-L round bottom flask equipped with a Teflon® stir blade, thermocouple, addition funnel, pH probe, condenser and nitrogen inlet was charged with a dispersion of the IEP 2.0 silica-coated red iron oxide pigment containing 10% solids and having a particle size distribution wherein 50% of the particles had a diameter of less than 475 nm. The pH of the mixture was adjusted to 2.86 with sulfuric acid, 3.6 g of aluminum sulfate octadecahydrate was added, and the mixture was stirred at room temperature for five minutes. Then, 6.0 mL of 28% ammonium hydroxide solution was diluted into 114 mL of water and 81 g of this mixture was added over two hours to the dispersion with stirring. Over this time the pH of the dispersion rose from 2.86 to 8.1. After this time, the dispersion was ultrafiltered with a total of 6 additional liters of water to a conductivity of 0.432 mS to yield 1148 g of a dispersion containing 18.6% solids, having a particle size distribution wherein 50% of the particles had a diameter of less than 423 nm, and an IEP of 5.11. A portion of this dispersion was pH adjusted to 4.9 with sulfuric acid and the mixture was filtered through a coarse glass frit. The filter cake was dried overnight under vacuum to give 145.2 g of the silica-coated red iron oxide pigment having an IEP of 5 as a red solid.

Analysis of the silica-coated red iron oxide pigment using ESCA indicated that the surface concentration of silicon was 11.9 atom percent, and the surface concentration of iron was 18.6 atom percent. The unfunctionalized red iron oxide pigment as received was also analyzed by ESCA and was found to have a surface concentration of silicon and iron of 2.5 atom percent and 39.6 atom percent respectively.

### Example 2

### Preparation of Isocyanate-Functionalized Silica-Coated Red Iron Oxide Pigment

This Example illustrates the preparation of isocyanate-functionalized silica-coated red iron oxide pigment. A dispersion of silica-coated red iron oxide pigment was reacted with 3-isocyanatopropyl-triethoxysilane to form the isocyanate-functionalized pigment. Silica-coated red iron oxide pigment having an IEP of 4.9 (2.5 g), prepared as described in Example 1, was suspended in 30 mL of dry tetrahydrofuran in a 50 mL plastic centrifuge tube. The pigment suspension was sonicated for 1 min on a Branson Sonifier® 150 (Branson Ultrasonics Corp., Danbury, Conn.) at a power setting of 6. Then, 0.395 g (1.6 mmol) of 3-isocyanatopropyl-triethoxysilane was added to the pigment suspension in a dry box. This entire procedure was repeated three times to provide a total of 10 g of starting pigment. The tubes were capped and sealed with plastic tape and mixed on a vortex mixer (Model VX-2500, VWR Scientific, West Chester, Pa.) at the lowest speed setting. The tubes were then centrifuged at 4000 rpm for 10 min to pellet the functionalized pigment and the supernatant was decanted. An additional 30 mL of dry tetrahydrofuran was added to each tube, the tubes were mixed on the vortex mixer, and centrifuged at 4000 rpm for 10 min, after which the supernatant was decanted. This wash step was repeated two more times and the final product was dried under vacuum at room temperature to yield 9.1 g of the isocyanate-functionalized silica-coated red iron oxide pigment as an orange-red powder.

Analysis of the isocyanate-functionalized silica-coated red iron oxide pigment using ESCA indicated that the surface concentration of silicon was 12.5 atom percent, and the surface concentration of iron was 17.1 atom percent.

### Example 3

### Preparation of Isocyanate-Functionalized Red Iron Oxide Pigment

This Example illustrates the preparation of isocyanate-functionalized red iron oxide pigment. A dispersion of red iron oxide pigment was reacted with 3-isocyanatopropyl-triethoxysilane to form the isocyanate-functionalized pigment.

Red iron oxide pigment, (Sensient LC381, 2.5 g) was suspended in 30 mL of dry toluene in a 50 mL plastic centrifuge tube. The contents of the tube were sonicated for 2 min on a Branson Sonifier® 150 at a power setting of 6. Then, 3-isocyanatopropyltriethoxysilane (0.395 g, 1.6 mmol) was added to the pigment dispersion in a dry box. The above procedure was repeated three times to provide a total of 10 g of starting pigment. The tubes were capped sealed with plastic tape and mixed overnight at room temperature on a vortex mixer (Model VX-2500, VWR Scientific) at the lowest speed setting. The tubes were then centrifuged at 4000 rpm for 10 min to pellet the functionalized pigment and the supernatant was decanted. An additional 30 mL of dry toluene was added to each tube, the resulting suspension was mixed on the vortexer, and then centrifuged at 4000 rpm for 10 min, after which time the supernatant was decanted. This wash step was repeated two more times and the final product was dried under vacuum at room temperature to yield 9 g of the isocyanate-functionalized red iron oxide pigment as an orange-red powder with an average particle size of 663 nm.

Analysis of the isocyanate-functionalized red iron oxide pigment using ESCA indicated that the surface concentration of silicon was 3.8 atom percent, and the surface concentration of iron was 30.1 atom percent.

### Example 4

### Preparation of a Peptide-Based Coloring Reagent Comprising Hair-Binding Peptide Gray3-K₅ Covalently Bound to an Isocyanate-Functionalized Silica-Coated Pigment

This Example illustrates the covalent attachment of an isocyanate-functionalized silica-coated red iron oxide pigment to a hair-binding peptide.

The hair-binding peptide Gray3-K₅, given as SEQ ID NO:230, was synthesized using Merrifield methods by SynBioSci (Livermore, Calif.) and obtained in >70% purity after purification by high performance liquid chromatography (HPLC). The peptide (50 mg, 0.0202 mmol) was dissolved in 10 mL of freshly dried dimethylformamide (DMF) to yield a clear solution. Triethylamine (20 mg, 0.2 mmol) was added and the mixture was shaken vigorously in a nitrogen-filled dry box. Then, 500 mg of isocyanate functionalized, silica-coated red iron oxide pigment, prepared as described in Example 2, was added and the mixture was sonicated for 1 min on a Branson Sonifier0 150 at a power setting of 6. The sealed reaction tube was then placed on the vortex mixer and mixed for 6 h at room temperature. After 6 h the peptide/pigment adduct was collected by centrifugation, washed in deionized water, collected again by centrifugation and finally dried under vacuum at 60° C. to yield 468 mg of the peptide-based coloring reagent as an orange-red powder.

### Example 5

### Preparation of a Peptide-Based Coloring Reagent Comprising Hair-Binding Peptide HP2-K₅ Covalently Bound to an Isocyanate-Functionalized Silica-Coated Pigment

This Example illustrates the covalent attachment of an isocyanate-functionalized silica-coated red iron oxide pigment to a hair-binding peptide.

The hair-binding peptide HP2-K₅, given as SEQ ID NO:231, was synthesized using Merrifield methods by SynBioSci (Livermore, Calif.) and obtained in >70% purity after HPLC purification. The peptide (50 mg, 0.017 mmol) was dissolved in 10 mL of freshly dried dimethylformamide to yield a clear solution. Triethylamine (20 mg, 0.2 mmol) was added and the mixture was shaken vigorously in a nitrogen-filled dry box. Then, 500 mg of isocyanate functionalized, silica-coated red iron oxide pigment, prepared as described in Example 2, was added and the mixture was sonicated for 1 min on a Branson Sonifier® 150 at a power setting of 6. The sealed reaction tube was then placed on the vortex mixer and mixed for 6 h at room temperature. After 6 h the peptide/pigment adduct was collected by centrifugation, washed in deionized water, collected again by centrifugation and finally dried under vacuum at 60° C. to yield 430 mg of the peptide-based coloring reagent as an orange-red powder.

### Example 6

### Preparation of a Peptide-Based Coloring Reagent Comprising Isocyanate-Functionalized Hair-Binding Peptide HP2-K₅ Covalently Bound to a Red Iron Oxide Pigment

This Example illustrates the covalent attachment of red iron oxide pigment to a hair-binding peptide. The peptide was first reacted with 3-isocyanatotriethoxysilane to form a functionalized peptide, which was then reacted with the red iron oxide pigment.

The hair-binding peptide HP2-K5, given as SEQ ID NO:231, was synthesized using Merrifield methods by SynBioSci (Livermore, Calif.) and obtained in >70% purity after HPLC purification. The peptide (60 mg, 0.02 mmol) was dissolved in 20 mL of freshly dried dimethylformamide to yield a clear solution. Then, 3-isocyanatotriethoxysilane (5 mg, 0.02 mmol) and triethylamine (20 mg, 0.2 mmol) was added and the mixture was shaken vigorously in a nitrogen-filled dry box and stirred for 24 h at room temperature. After confirming that silane addition to the peptide had occurred using MALDI mass spectrometry, 800 mg of red iron oxide pigment (Sensient LC381) was added and the mixture was sonicated for one minute on a Branson Sonifier® 150 at a power setting of 6 . The sealed reaction tube was then placed on the vortex mixer and mixed for 12 h at room temperature. The peptide-based coloring reagent was collected by centrifugation, washed twice in deionized water, collected again by centrifugation and dried.

### Example 7

### Preparation of a Peptide-Based Coloring Reagent Comprising Isocyanate-Functionalized Hair-Binding Peptide Gray3-K₅ Covalently Bound to a Red Iron Oxide Pigment

This Example illustrates the covalent attachment of red iron oxide pigment to a hair-binding peptide. The peptide was first reacted with 3-isocyanatotriethoxysilane to form a functionalized peptide, which was then reacted with the red iron oxide pigment.

The hair-binding peptide Gray3-K₅, given as SEQ ID NO:230, was synthesized using Merrifield methods by SynBioSci (Livermore, Calif.) and obtained in >70% purity after HPLC purification. The peptide (62 mg, 0.025 mmol) was dissolved in 20 mL of freshly dried dimethylformamide (DMF) to yield a clear solution. Then, 3-isocyanatotriethoxysilane (5 mg, 0.02 mmol) and triethylamine (20 mg, 0.2 mmol) were added and the mixture was shaken vigorously in a nitrogen-filled dry box and stirred for 24 h at room temperature. After this time the DMF was removed by evaporation and the residue was resuspended in N-methylpyrrolidinone (20 mL) with the addition of an additional 5 mg of 3-isocyanatopropyltriethoxysilane. After confirming that silane addition to the peptide had occurred using MALDI mass spectrometry, 800 mg of red iron oxide pigment (Sensient LC 381) was added and the mixture was sonicated for one minute on a Branson Sonifier®150 at a power setting of 6. The sealed reaction tube was then placed on the vortex mixer and mixed for 12 h at room temperature. The peptide-based coloring reagent was collected by centrifugation, washed twice in deionized water, collected again by centrifugation and dried.

### Example 8

Preparation of a Peptide-Based Coloring Reagent Comprising Hair-Binding Peptide HP2-C Covalently Bound to an Isocyanate-Functionalized Pigment

This Example illustrates the covalent attachment of an isocyanate-functionalized red iron oxide pigment to a hair-binding peptide.

The hair-binding peptide, HP2-C, given as SEQ ID NO:232, was synthesized using Merrifield methods by SynBioSci (Livermore, Calif.) and obtained in >70% purity after HPLC purification. The peptide (30 mg, 0.012 mmol) was suspended in 10 mL of acetonitrile and 3-mercaptopropyltrimethoxysilane (4.9 mg, 0.024 mmol) was added. Then, triethylamine (12.5 mg) was added and the mixture was stirred under nitrogen at 55° C. for 12 h. After this time, dimethylformamide (10 mL) was added to the reaction mixture along with 400 mg of isocyanate functionalized red iron oxide prepared as in Example 3. The mixture were stirred for 24 h at room temperature and the peptide/pigment adduct was collected by centrifugation. The product was washed with fresh DMF, collected by centrifugation and dried under vacuum at 60° C. to yield the peptide-based coloring reagent as an orange-red powder.

### Example 9

### Preparation of a Peptide-Based Coloring Reagent Comprising Hair-Binding Peptide Gray5-C Covalently Bound to an Isocyanate-Functionalized Pigment

This Example illustrates the covalent attachment of an isocyanate-functionalized red iron oxide pigment to a hair-binding peptide.

The hair-binding peptide Gray5-C, given as SEQ ID NO:233, was synthesized using Merrifield methods by SynBioSci (Livermore, Calif.) and obtained in >70% purity after HPLC purification. The peptide (30 mg, 0.0081 mmol) was suspended in 10 mL of acetonitrile and 3-mercaptopropyltrimethoxysilane (3.1 mg, 0.016 mmol) was added. Then, triethylamine (8.15 mg) was added and the mixture was stirred under nitrogen at 55° C. for 12 h. After this time, dimethylformamide (10 mL) was added to the reaction mixture along with 400 mg of isocyanate functionalized red iron oxide prepared as described in Example 3. The mixture was stirred for 24 h at room temperature and the peptide/pigment adduct was collected by centrifugation. The product was washed with fresh DMF, collected by centrifugation and dried under vacuum at 60° C. to yield the peptide-based coloring reagent as an orange-red powder.

### Example 10

### Preparation of a Peptide-Based Coloring Reagent Comprising Hair-Binding Peptide CXHG102-C Covalently Bound to an Isocyanate-Functionalized Pigment

This Example illustrates the covalent attachment of an isocyanate-functionalized red iron oxide pigment to a hair-binding peptide.

The hair-binding peptide CXHG102-C, given as SEQ ID NO:234, was synthesized using Merrifield methods by SynBioSci (Livermore, CA) and obtained in >70% purity after HPLC purification. The peptide (30 mg, 0.012 mmol) was suspended in 10 mL of acetonitrile and 3-mercaptopropyltrimethoxysilane (4.9 mg, 0.024 mmol) was added. Then, triethylamine (12.6 mg) was added and the mixture was stirred under nitrogen at 55° C. for 12 h. After this time, dimethylformamide (10 mL) was added to the reaction mixture along with 400 mg of isocyanate functionalized red iron oxide prepared as described in Example 3. The mixture was stirred for 24 h at room temperature and the peptide/pigment adduct was collected by centrifugation. The product was washed with fresh DMF, collected by centrifugation and dried under vacuum at 60° C. to yield the peptide-based coloring reagent as an orange-red powder.

### Examples 11-21

### Coloring Hair with Peptide-Based Coloring Reagents

These Examples illustrate the coloring of hair using peptide-based coloring reagents. The durability of the hair coloring was evaluated using a bead embrocation shampoo and washing procedure

Human natural white hair was obtained from International Hair Importers and Products (Bellerose, NY) and cut into 2.5 cm long×0.8 cm wide tresses that were potted on one end with Scotch-Grip™4475 plastic adhesive (3M, St. Paul, Minn.). An effort was made to restrict the tress samples to a portion of the middle of 4-6 inch (10-15 cm) long tresses as received from the supplier to minimize possible bias from root and tip variations. The tresses were soaked in deionized water for at least 30 min prior to use.

The peptide-based coloring reagents (50 mg), prepared as described in Examples 4-10, were suspended in 13 mL of deionized water containing 50 mg of thioglycolic acid (TGA) in separate 15 mL plastic centrifuge tubes. For some hair coloring experiments, the TGA adjuvant was omitted, as indicated in Table 1. The suspensions were sonicated twice for one minute on a Branson Sonifier®150 at a power level of 6. Two small natural white tresses (prepared as described above) were then immersed in each colorant suspension and mixed on a vortex mixer at the lowest power setting and a 50% duty cycle for 4 h. The colored tresses were then removed and rinsed under flowing deionized water and allowed to air dry. This hair coloring procedure was repeated using 50 mg of the isocyanate-functionalized, silica-coated red iron oxide pigment described in Example 2, or the isocyanate-functionalized red iron oxide pigment described in Example 3 in place of the peptide-based coloring reagent to serve as controls.

The durability of the hair coloring was evaluated using a bead embrocation shampoo and washing procedure. The tresses were added to the wells of a 24-well plate and subjected to shampoo cycles. Beads were added to each well at the beginning of a cycle as follows: four 3 mm glass beads, one 4 mm stainless steel bead, and two 6.35 mm glass beads. Approximately 1.0 mL of a 0.2% sodium lauryl ether sulfate (SLES) solution was added to each well. The well plate was covered with a flexible SANTOPRENE® mat and was agitated at high speed on a vortex mixer for 30 sec. The shampoo was removed from the wells by suction. Approximately 4 mL of de-ionized water was added to each well, the plate was agitated at a low speed on the vortex mixer for 5-10 sec, and the rinse solution was removed by suction. The tresses were thoroughly rinsed under a jet of de-ionized water and subjected to the next shampoo cycle. After the fifth shampoo cycle, the tresses were dried in air.

Color intensity after water rinse or shampoo washing was measured using an X-Rite® SP78™ Sphere Spectrophotometer (X-Rite, Inc., Grandville, Mich.), by placing the colored hair sample into the photosensor and calculating L*, a* and b* parameters representing the photometer response. An initial baseline L* value was measured for the uncolored hair and all measurements were the average of three individual determinations. In this case the Delta E values were indicative of color retention after shampoo or water rinse treatments and large Delta E values indicate better performance.

Delta E values were calculated from L*, a*, and b* using the formulae:

Delta E Uptake=((Lu*−L0)²+(au*−a0*)²+(bu*−b0*)²)^{1/2}

Delta E retention=((Lr*−L0)²+(ar*−a0*)²+(br−b0*)²)^{1/2}

where,
Lu*, au* and bu* are L*, a* and b* values for a sample tress after color uptake,
Lr*, ar* and br* are L*, a* and b* values for a sample tress after shampoo cycles, and
L0*, a0* and b0* are L*, a* and b* values for untreated natural white hair
(L*=the lightness variable and a* and b* are the chromaticity coordinates of CIELAB colorspace as defined by the International Commission of Illumination (CIE) (Minolta, Precise Color Communication—Color Control From Feeling to Instrumentation, Minolta Camera Co., 1996).

The results of the color intensity measurements are summarized in Table 1. As shown in the table, the peptide-based coloring reagents provided higher color uptake as compared to the pigment only controls (Comparative Examples 11, 14, and 17) and gave enhanced colorant retention after 5 shampoo wash cycles carried out with bead embrocation as described above.

TABLE 1
Results of Color Intensity Measurements After Shampooing and Washing

| | | Delta E | Delta E | |
|---|---|---|---|---|
| Example | Colorant | Uptake | Retention | Adjuvant |
| 11, | isocyanate- | 28 | 17 | TGA |
| Comparative | functionalized | | | |
| | silica-coated red | | | |
| | iron oxide | | | |
| | pigment from | | | |
| | Example 2 | | | |
| 12 | peptide-based | 30 | 18 | TGA |
| | coloring reagent | | | |
| | from Example 4 | | | |
| 13 | peptide-based | 32 | 20 | TGA |
| | coloring reagent | | | |
| | from Example 5 | | | |
| 14, | isocyanate- | 31 | 19 | None |
| Comparative | functionalized | | | |
| | red iron oxide | | | |
| | pigment coloring | | | |
| | reagent from | | | |
| | Example 3 | | | |
| 15 | peptide-based | 38 | 24 | None |
| | coloring reagent | | | |
| | from Example 8 | | | |
| 16 | peptide-based | 38 | 23 | None |
| | coloring reagent | | | |
| | from Example | | | |
| | 10 | | | |
| 17, | isocyanate- | 31 | 15 | TGA |
| Comparative | functionalized | | | |
| | red iron oxide | | | |
| | pigment coloring | | | |
| | reagent from | | | |
| | Example 3 | | | |
| 18 | peptide-based | 36 | 25 | TGA |
| | coloring reagent | | | |
| | from Example 9 | | | |
| 19 | peptide-based | 32 | 21 | TGA |
| | coloring reagent | | | |
| | from Example 7 | | | |
| 20 | peptide-based | 39 | 25 | TGA |
| | coloring reagent | | | |
| | from Example 6 | | | |
| 21 | peptide-based | 36 | 22 | TGA |
| | coloring reagent | | | |
| | from Example 8 | | | |

What is claimed is:

1. A peptide-based coloring reagent selected from the group consisting of:
a) (BSBP)ₙ—CP; and
b) [(BSBP)ₘ—S]ₙ—CP;
wherein:
(i) BSBP is a body surface-binding peptide;
(ii) CP is a coated pigment containing at least 3 atom percent of silicon on its surface, as determined by electron spectroscopy for chemical analysis (ESCA);
(iii) S is a molecular spacer;
(iv) BSBP is covalently bound to the surface of CP in (a) and S is covalently bound to the surface of CP in (b);
(v) m ranges from 1 to about 50; and
(vi) n ranges from 1 to about 100,000.

2. A peptide-based coloring reagent according to claim 1 wherein the body surface-binding peptide is selected from the group consisting of; a hair-binding peptide, a skin-binding peptide, a nail-binding peptide, and a tooth-binding peptide

3. A peptide-based coloring reagent according to claim 1 wherein the coated pigment contains less than about 40 atom percent of metal atoms on its surface.

4. A peptide-based coloring reagent according to claim 1 wherein the coated pigment a coated metal oxide.

5. A peptide-based coloring reagent according to claim 4 wherein the coated metal oxide is iron oxide.

6. A peptide-based coloring reagent according to claim 5 wherein the iron oxide comprises isocyanate or sulfhydryl functional groups.

7. A peptide-based coloring reagent according to claim 1 wherein the molecular spacer is selected from the group consisting of a peptide spacer and an organic spacer

8. A peptide-based coloring reagent according to claim 1 wherein the coated pigment is coated with silica.

9. A peptide-based coloring reagent according to claim 1 wherein the coated pigment is coated with at least one silane coupling reagent.

10. A peptide-based coloring reagent according to claim 9 wherein the silane coupling reagent is selected from the group consisting of isocyanatopropylsilane, mercaptopropylsilane, aminopropylsilane, 3-chloropropyltrimethoxysilane, 3-chloropropyltriethoxysilane, 3-chloropropylmethyldimethoxysilane, 3-chloropropylmethyldiethoxysilane, vinyltrimethoxysilane, methacryloxypropyltrimethoxysilane, vinyltriethoxysilane, and combinations thereof.

11. A personal care composition comprising at least one peptide-based coloring reagent selected from the group consisting of:
a) (BSBP)ₙ—CP; and
b) [(BSBP)ₘ—S]ₙ—CP;
wherein:
(i) BSBP is a body surface-binding peptide;
(ii) CP is a coated pigment containing at least 3 atom percent of silicon on its surface, as determined by electron spectroscopy for chemical analysis (ESCA);
(iii) S is a molecular spacer;
(iv) BSBP is covalently bound to the surface of CP in (a) and S is covalently bound to the surface of CP in (b);
(v) m ranges from 1 to about 50; and
(vi) n ranges from 1 to about 100,000.

12. A personal care composition according to claim 11 wherein the personal care composition is a hair care or hair coloring composition and the body surface-binding peptide is selected from the group consisting of a hair-binding peptide, a skin-binding peptide, a nail-binding peptide and a tooth-binding peptide

13. A method for coloring a body surface comprising: applying a personal care composition comprising at least one peptide-based coloring reagent selected from the group consisting of:
a) (BSBP)ₙ—CP; and
b) [(BSBP)ₘ—S]ₙ—CP;
wherein:
(i) BSBP is a body surface-binding peptide;
(ii) CP is a coated pigment containing at least 3 atom percent of silicon on its surface, as determined by electron spectroscopy for chemical analysis (ESCA);
(iii) S is a molecular spacer;
(iv) BSBP is covalently bound to the surface of CP in (a) and S is covalently bound to the surface of CP in (b);
(v) m ranges from 1 to about 50; and
(vi) n ranges from 1 to about 100,000;
to the body surface for a time sufficient for the peptide-based coloring reagent to bind to the body surface.

14. A method according to claim 13 wherein the body surface is selected from the group consisting of hair, skin, nails, and teeth.
